(19) United States
(12) Patent Application Publication
(10) Pub. No.: US 2013/0202629 A1
(43) Pub. Date: Aug. 8, 2013

(54) USES OF PHOSPHOLIPID CONJUGATES OF SYNTHETIC TLR7 AGONISTS

(71) Applicants: Dennis A. Carson, La Jolla, CA (US); Howard B. Cottam, Escondido, CA (US); Tomoko Hayashi, San Diego, CA (US); Michael Chan, San Diego, CA (US); Christina C.N. Wu, Escondido, CA (US)

(72) Inventors: Dennis A. Carson, La Jolla, CA (US); Howard B. Cottam, Escondido, CA (US); Tomoko Hayashi, San Diego, CA (US); Michael Chan, San Diego, CA (US); Christina C.N. Wu, Escondido, CA (US)

(73) Assignee: The Regents of the University of California, Oakland, CA (US)

(21) Appl. No.: 13/695,385

(22) PCT Filed: Apr. 29, 2011
(86) PCT No.: PCT/US11/00757

§ 371 Date: Feb. 12, 2013

Related U.S. Application Data
(60) Provisional application No. 61/343,573, filed on Apr. 30, 2010.

(51) Int. Cl. A61K 39/39; C07F 9/6561

(52) U.S. Cl. CPC A61K 39/39; C07F 9/65616
USPC 424/184.1

(57) ABSTRACT

The invention provides uses for phospholipid conjugates of TLR agonists, for instance in vaccines, and to prevent, inhibit or treat a variety of disorders including inflammation, cancer and pathogen, e.g., microbe, infection.

### CROSS-REFERENCE TO RELATED APPLICATIONS

This application claims the benefit of the filing date of U.S. provisional application Ser. No. 61/343,573, filed on Apr. 30, 2010, the disclosure of which is incorporated by reference herein.

### STATEMENT OF GOVERNMENT RIGHTS

The invention described herein was made with government support under Grant Numbers AI056453 and AI077989 awarded by the National Institutes of Health. The United States Government has certain rights in the invention.

### BACKGROUND

A great deal has been learned about the molecular basis of innate recognition of microbial pathogens in the last decade. It is generally accepted that many somatic cells express a range of pattern recognition receptors that detect potential pathogens independently of the adaptive immune system (see Janeway et al., Annu. Rev. Immunol., 20:197 (2002)). These receptors are believed to interact with microbial components termed pathogen associated molecular patterns (PAMPs). Examples of PAMPs include peptidoglycans, lipotechoic acids from gram-positive cell walls, the sugar mannose (which is common in microbial carbohydrates but rare in humans), bacterial DNA, double-stranded RNA from viruses, and glucans from fungal cell walls. PAMPs generally meet certain criteria that include (a) their expression by microbes but not their mammalian hosts, (b) conservation of structure across the wide range of pathogens, and (c) the capacity to stimulate innate immunity. Toll-like Receptors (TLRs) have been found to play a central role in the detection of PAMPs and in the early response to microbial infections (see Underhill et al., Curr. Opin. Immunol., 14:103 (2002)).

Ten mammalian TLRs and a number of their agonists have been identified. For example, guanine and uridine-rich single-stranded RNA has been identified as a natural ligand for TLR7 (Diebold et al., Science, 303:1529 (2004)). In addition, several low molecular weight activators of TLR7 have been identified, including imidazoquinolines, and purine-like molecules (Hemmi et al., Nat. Immunol., 3:191 (2002); Lee et al., Proc. Natl. Acad. Sci. USA, 180:6646 (2003); Lee et al., Nat. Cell Biol., 8:1327 (2006)). Among the latter, 9-benzyl-8-hydroxy-2-(2-methoxyethoxy) adenine (“SM”), has been identified as a potent and specific TLR7 agonist. The synthetic immunomodulator R-848 (resiquimod) activates both TLR7 and TLR8. While TLR stimulation initiates a common signaling cascade (involving the adaptor protein MyD88, the transcription factor NF-kB, and pro-inflammatory and effector cytokines), certain cell types tend to produce certain TLRs. For example, TLR7 and TLR9 are found predominantly on the internal faces of endosomes in dendritic cells (DCs) and B lymphocytes (in humans; mouse macrophages express TLR7 and TLR9). TLR8, on the other hand, is found in human blood monocytes (see Hornung et al., J. Immunol., 168:4531 (2002)).

### SUMMARY OF THE INVENTION

The present invention provides uses for a synthetic TLR7 agonist linked via a stable covalent bond to a phospholipid macromolecule (a conjugate), i.e., the conjugate does not act as a prodrug. The conjugates may include phospholipid macromolecules directly linked to a synthetic TLR7 agonist or linked via a linker to the TLR7 agonist, for instance, linked via an amino group, a carboxy group or a succinamide group. The conjugates of the invention are broad-spectrum, long-lasting, and non-toxic synthetic immunostimulatory agents, which are useful for activating the immune system of a mammal, e.g., a human, in vivo by stimulating the activity of TLR7. In particular, the conjugates of the invention optimize the immune response while limiting undesirable systemic side effects associated with unconjugated TLR7 agonists.

Thus, the invention provides methods of augmenting an immune response, e.g., an immune response to a specific antigen, or inducing a general immune response (in the absence of a specific antigen). In one embodiment, the conjugate acts as an adjuvant and so is associated with a specific not a general immune response. In one embodiment, the conjugate acts as a general immune stimulator. In one embodiment, the method includes administering to a mammal in need thereof an amount of an antigen and a conjugate of the invention effective to prevent, inhibit or treat disorders, including but not limited to microbial infections, bladder conditions or skin conditions. Non-limiting examples of antigens useful in the invention include but are not limited to isolated proteins or peptides, e.g., dipeptides or tripeptides, and the like; carbohydrates (polysaccharides), nucleotides such as, for example, PNA, RNA and DNA, and the like; cells, lipids, microbes, for example, viruses, bacteria, fungi, and the like. The antigens can include inactivated whole organisms or microbes, or sub-components thereof and the like. In one embodiment, the immune response to the administration of the antigen and the conjugate is enhanced relative to the administration of the antigen (in the absence of the conjugate) or a corresponding unconjugated TLR7 agonist, or a combination thereof. In one embodiment, a mammal is administered a composition comprising the antigen and the conjugate. In one embodiment, the composition is locally administered, e.g., dermal or intranasal administration. In another embodiment, the composition is systemically administered. In another embodiment, the antigen and conjugate are formulated separately and administered concurrently or sequentially.

The invention thus provides immunogenic compositions comprising an amount of a conjugate of the invention, for instance, one that alone induces an inflammatory response, and also is effective to augment an immune response to an antigen. In one embodiment, the composition does not include a solvent or preservative such as DMSO or ethanol, which may have toxic effects, e.g., in humans.

A conjugate of the invention has formula (I):

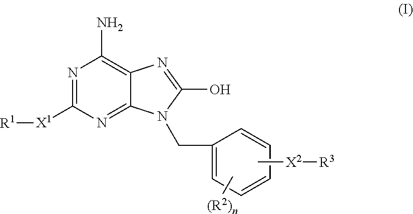

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{c} is hydrogen, C₁₋₁₀alkyl, or substituted C₁₋₁₀alkyl; or R^{C} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R³ is a phospholipid comprising one or two carboxylic esters; or a tautomer thereof;

or a pharmaceutically acceptable salt or solvate thereof.

In one embodiment, the composition of the invention comprises nanoparticles comprising a compound of formula (I). As used herein, a nanoparticle has a diameter of about 30 nm to about 600 nm, or a range with any integer between 30 and 600, e.g., about 40 nm to about 250 nm, including about 40 to about 80 or about 100 nm to about 150 nm in diameter. The nanoparticles may be formed by mixing a compound of formula (I), which may spontaneously form nanoparticles, or by mixing a compound of formula (I) with a preparation of lipids, such as phospholipids including but not limited to phosphatidylcholine, phosphatidylserine or cholesterol, thereby forming a nanoliposome. Optionally, a compound of formula (I), a lipid preparation and a glycol such as propylene glycol are combined.

In one embodiment, a single dose of the conjugate may show very potent activity in stimulating the immune response. Moreover, because of the low toxicity of the conjugates, in some circumstances higher doses may be administered, e.g., systemically, while under other circumstances lower doses may be administered, e.g., due to localization of the conjugate.

The invention thus provides a conjugate of the invention for use in medical therapy, e.g., in a vaccine for prophylaxis of microbial infections, such as bacterial or viral infections, as well as for the manufacture of a medicament for the treatment of a TLR7 associated condition or symptom in which an augmented immune response is indicated, e.g., cancer. Bacterial infections include but are not limited to Staphylococcus, Streptococcus, Enterococcus, Bacillus, Corynebacterium, Nocardia, Clostridium, Actinobacteria, Listeria, and Actinobacteria; Mycoplasma; Escherichia coli, Salmonella, Shigella, and other Enterobacteriaceae, Mycobacteria, Pseudomonas, Moraxella, Helicobacter, Stenotrophomonas, Bdellovibrio, acetic acid bacteria, and Legionella and including Neisseria gonorrhoeae, Neisseria meningitidis, Mycobacterium tuberculosis, Mycobacterium leprae, Moraxella catarrhalis, Hemophilus influenzae, Klebsiella pneumoniae, Legionella pneumophila, Pseudomonas aeruginosa, Proteus mirabilis, Enterobacter cloacae, Serratia marcescens, Helicobacter pylori, Salmonella enteritidis, Salmonella typhi, and Acinetobacter baumannii infections. Viral infections include but are not limited to lentivirus, retrovirus, coronavirus, influenza virus, hepatitis virus, rhinovirus, papilloma virus, herpes virus or influenza virus infections. The conjugates of the invention can also be used for biodefense, e.g., against B. anthrax.

As described hereinbelow, a single administration of conjugate of the invention and irradiated Bacillus anthracis spores to mice 6 days before challenge prolonged survival, while multiple administrations resulted in 100% survival 30 days after challenge.

Thus, in one embodiment, the invention provides a method to prevent, inhibit or treat a bacterial infection, for instance, gram-positive bacterial infection, in a mammal such as a human, bovine, equine, swine, canine, ovine, or feline. The method includes administering to the mammal an effective amount of a composition comprising a bacterial antigen and an amount of a compound having formula (I):

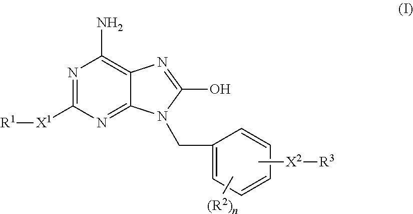

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R³ is a phospholipid comprising one or two carboxylic esters; or a tautomer thereof;

or a pharmaceutically acceptable salt or solvate thereof.

In addition, the invention also provides a pharmaceutical composition comprising at least one phospholipid conjugate of the invention, or a pharmaceutically acceptable salt thereof, in combination with a pharmaceutically acceptable diluent or carrier, optionally in combination with a preparation of a selected antigen, such as an antigen of a microbe, e.g., a killed preparation or an extract, isolated protein of a selected microbe, or isolated carbohydrate (polysaccharide) of a selected microbe. In one embodiment, a pharmaceutical composition comprises nanoparticles formed by combining at least one phospholipid conjugate of the invention, or a pharmaceutically acceptable salt thereof, in an aqueous solvent, e.g., PBS, or by combining at least one phospholipid conjugate of the invention, or a pharmaceutically acceptable salt thereof, and a preparation of phospholipids, e.g., in an aqueous solvent.

### BRIEF DESCRIPTION OF FIGURES

FIG. 1 illustrates the scheme used for synthesis of lipid- (6), PEG- (8), or lipid-PEG (9) TLR7 conjugates. (6), (8), and (9) refer to compound designations.

FIGS. 2A-D depict the results of in vitro immunological characterization of TLR7 conjugates in murine macrophages. In general, RAW264.7 cells (1×10⁶/mL) (A), and BMDM (0.5×10⁶/mL) from wild-type (B-D) or TLR7 deficient mice (C-D) were incubated with serial dilutions of conjugates for 18 hours at 37° C., 5% CO₂ and culture supernatants were collected. Serial dilutions of SM (triangle), 6 (gray circle), 8 (solid square), and 9 (ex) were prepared (steps of 1:5) starting from 10 μM; 4a (gray rhombus) was serially diluted (steps of 1:5) starting from 0.1 μM. The levels of cytokines (IL-6, IL-12 or TNF-alpha) in the supernatants were determined by ELISA (BD Biosciences Pharmingen, La Jolla, Calif.). Data are means±SEM of triplicates and are representative of three independent experiments. P<0.001 by two-way ANOVA with Bonferroni post hoc test in comparison between SM and 6. Denotes P<0.01 by Student's t-test compared to the corresponding data of wild-type macrophages.

FIGS. 3A-B depict the results of in vitro immunological characterization of TLR7 conjugates in human PBMC. Human PBMC (2×10⁶/mL) were incubated with serial dilutions of conjugates for 18 hours. Serial dilutions of SM (triangle), 6 (gray circle), 8 (solid square), and 9 (ex) were prepared (steps of 1:5) starting from 10 μM; 4a (gray rhombus) was serially diluted (steps of 1:5) stating from 0.1 μM. The levels of IL-6 (A) and IFNalpha1 (B) in the culture supernatants were determined by Luminex assay in duplicate. Data are means±SEM and are representative of three independent experiments. The order of potency is 4b>6>9>SM=8 (P<0.0005 by two-way ANOVA test for compounds 4b, 6, 9, and SM; P=0.2 by two-way ANOVA test for compound 8.

FIGS. 4A-B illustrate the kinetics of pro-inflammatory cytokine induction by TLR7 conjugates in vivo. C57BL/6 mice (n=5 per group) were intravenously injected with TLR7 conjugates (SM, 200 nmol: 4a, 40 nmol; 6, 200 nmol; 8, 200 nmol; 9, 200 nmol). Serum samples were collected 2, 4, 6, 24, and 48 hours after injection. The levels of TNF-alpha (A) and IL-6 (B) were measured by Luminex assay. Data are means±SEM of five mice and are representative of two independent experiments. (C) Control naïve mice. * denotes p<0.05 compared to naïve mice by one-way ANOVA tests with Bonferroni post hoc testing.

FIGS. 5A-C illustrate the adjuvanicity (e.g., ability to initiate an immunological response) of TLR7 conjugates in vivo. Groups of C57BL/6 mice (n=5 per group) were subcutaneously immunized with 20 μg OVA mixed with TLR7 conjugates (10 nmol equivalent dose per mouse) on days 0 and 7. Sera were collected days 0, 7, 14, 21, 28, 42, and 56. OVA specific IgG1 and IgG2a were measured by ELISA (A and B). On day 56, mice were sacrificed and splenocytes were cultured with OVA (100 μg/mL) in RPMI 1640 for 3 days. IFN-gamma level in the supernatant was measured by ELISA (C). Data are means±SEM of five mice/group and are representative of three independent experiments. * and ⁺ denote P<0.05 and P<0.01 by one-way ANOVA tests with Dunnett's post hoc comparison to mice immunized with OVA mixed with vehicle, respectively.

FIGS. 6A-C depict the results of evaluation of possible adverse effects of TLR7 conjugates. C57BL/6 mice were immunized with 20 μg OVA mixed with TLR7 conjugate. On day 56, mice were sacrificed and the number of total splenocytes was determined (A). The spleens were collected and submitted for histological examination (×100) (B). The skin at the site of injection was inspected 24 hours after injection (C). There was no significant difference in the splenocyte number between mice immunized with OVA plus TLR7 conjugates and mice immunized with OVA alone (A). Histological examination of the spleens from mice immunized with OVA mixed with TLR7 conjugates did not show any disruption of the white pulps or increased cellularity in red pulp (B). The skin at injection sites did not have any visible redness (C).

FIGS. 7A-B show time line of administration of and bladder inflammation induced by 1V270.

FIGS. 8A-D depict pharmacodynamics of cytokine induction by cream formulation of 1V270 compared to Aldara.

FIGS. 9A-F illustrate local cytokine induction in lung and serum after pulmonary administration of phospholipid conjugates.

FIGS. 10A-B depict the sustained local immune activation by a phospholipid conjugated TLR7 agonist.

FIGS. 11A-B show time line of single immunization and challenge, and the adjuvant effect of phospholipid conjugated TLR7 agonist.

FIGS. 12A-B show time line of multiple immunizations and challenge, and the adjuvant effect of phospholipid conjugated TLR7 agonist.

FIG. 13 illustrates the structure of UC-1V270 (compound 6 in FIG. 1).

FIG. 14 shows spontaneous nanoparticle formation of UC-1V270. UC-1V270 was diluted in PBS to 50 μM (A) or 100 μM (B) and particle size measured over time. The nanoparticles were generally stable over time. Some aggregates were seen at 100 μM which is about the upper limit of solubility. The particle size of UC-1V270 in PBS was relatively constant with an average diameter of about 110 nm regardless of concentration.

FIGS. 15A-F show UC-1V270 promotion of localized cytokine release with minimal systemic side effects. Four A/J mice were administered i.n. with UC-1V270, unconjugated TLR7 agonist (UC-1V209), phospholipid or a solvent control at various doses. BALF and plasma were collected 24 hours later and cytokine levels determined by multiplex Luminex assay. Bars indicate the mean.

FIGS. 16A-C illustrate complete long-term protection with UC-1V270 as an anthrax vaccine adjuvant. (A) Eight female A/J mice per group were administered i.n. with either PBS, irradiated spores (IRS) alone, UC-1V270 alone (1 nmol/mouse), IRS+UC-IV270 or IRS+CT (cholera toxin; 1 μg/mouse) three times at two week interval and challenged four weeks after the last immunization. (B) Survival was followed for 30 days. Kaplan-Meier survival curves and log-rank tests were performed to determined significance. Results were pooled from two separate experiments with a total of 16 mice per group. (C) Mice were sacrificed at 30 days after infection. Spleens were harvested and weighed.

FIGS. 17A-E show spore-specific Tₕ17 and Tₕ1 responses of surviving mice. Mice that survived infection after vaccination were sacrificed on day 30. Splenocytes (400,000/well) from those mice were cultured with IRS (10⁶/well) in triplicate for 5 days. Splenocytes from uninfected non-vaccinated mice served as a control. IL-12, IL-17 and TNF-α in the supernatant were measured by a Luminex assay. IFN-gamma was measured by ELISA. The data shown are pooled from two independent experiments.

FIG. 18 illustrates that depletion of IFN-gamma and IL-17 renders immunized mice susceptible to infection. Female A/J mice were administered i.n. with IRS+UC-1V270 (1 nmole/mouse) 3 times as indicated. Antibodies (0.2 mg anti-IL-17 and 0.1 mg anti-IFN gamma) were given twice daily starting one day prior to live anthrax spore challenge. Survival was followed for 24 days. Kaplan-Meier survival curves and log-rank tests were performed to determine significance.

FIG. 19 shows a comparison of cytokine levels in animals administered Phosal 50PG formulated UC-1V270 and UC-1V270 without Phosal 50PG (referred to as “unformulated”). An mice were administrated i.n. with UC-1V270 unformulated or Phosal 50PG formulated at doses indicated or vehicles (V1=5% DMSO, V2=1.2% Phosal 50PG). Plasma and BALF were collected 24 hours later and cytokine levels were analyzed by Luminex assay. Formulated UC-1V270 induced local cytokines more effectively compared to unformulated UC-1V270 with similar systemic cytokine induction, which is barely detectable except IFN-gamma.

FIG. 20 shows survival after infection in the anthrax model. A/J mice were intranasally immunized with either PBS or 2.5% Phosal 50PG vehicle alone, or with 5×10⁷ IRS in combination with various amounts of formulated UC-1V270 as indicated or CT. Mice were challenged with live anthrax spores 3 weeks after the last immunization (3 immunizations at 2 weeks intervals). All 3 doses of formulated UC-1V270 protected animals from anthrax infection. Note that in previous studies using unformulated 1V270 in DMSO, a 1 nmole dose was shown to be effective. In contrast, a 10 times lower dose of formulated UC-1V270 provided for high survival rates.

### DETAILED DESCRIPTION OF INVENTION

### Definitions

A composition is comprised of “substantially all” of a particular compound, or a particular form a compound (e.g., an isomer) when a composition comprises at least about 90%, and at least about 95%, 99%, and 99.9%, of the particular composition on a weight basis. A composition comprises a “mixture” of compounds, or forms of the same compound, when each compound (e.g., isomer) represents at least about 10% of the composition on a weight basis. A TLR7 agonist of the invention, or a conjugate thereof, can be prepared as an acid salt or as a base salt, as well as in free acid or free base forms. In solution, certain of the compounds of the invention may exist as zwitterions, wherein counter ions are provided by the solvent molecules themselves, or from other ions dissolved or suspended in the solvent.

As used herein, the term “isolated” refers to in vitro preparation, isolation and/or purification of a nucleic acid molecule, a peptide or protein, or other molecule so that it is not associated with in vivo substances or is present in a form that is different than is found in nature. Thus, the term “isolated” when used in relation to a nucleic acid, as in “isolated oligonucleotide” or “isolated polynucleotide” refers to a nucleic acid sequence that is identified and separated from at least one contaminant with which it is ordinarily associated in its source. An isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. Hence, with respect to an “isolated nucleic acid molecule”, which includes a polynucleotide of genomic, cDNA, or synthetic origin or some combination thereof, the “isolated nucleic acid molecule” (1) is not associated with all or a portion of a polynucleotide in which the “isolated nucleic acid molecule” is found in nature, (2) is operably linked to a polynucleotide which it is not linked to in nature, or (3) does not occur in nature as part of a larger sequence. The isolated nucleic acid molecule may be present in single-stranded or double-stranded form. When a nucleic acid molecule is to be utilized to express a protein, the nucleic acid contains at a minimum, the sense or coding strand (i.e., the nucleic acid may be single-stranded), but may contain both the sense and anti-sense strands (i.e., the nucleic acid may be double-stranded).

The term “amino acid” as used herein, comprises the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, -methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an -methylbenzyl amide). Other suitable amino and carboxy protecting groups are known to those skilled in the art (see for example, T. W. Greene, Protecting Groups In Organic Synthesis; Wiley: New York, 1981, and references cited therein). For instance, an amino acid can be linked to the remainder of a compound of formula I through the carboxy terminus, the amino terminus, or through any other convenient point of attachment, such as, for example, through the sulfur of cysteine.

The term “toll-like receptor agonist” (TLR agonist) refers to a molecule that binds to a TLR. Synthetic TLR agonists are chemical compounds that are designed to bind to a TLR and activate the receptor.

The term “nucleic acid” as used herein, refers to DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 7-position purine modifications, 8-position purine modifications, 9-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a BHQ, a fluorophore or another moiety.

A “phospholipid” as the term is used herein refers to a glycerol mono- or diester bearing a phosphate group bonded to a glycerol hydroxyl group with an alkanolamine group being bonded as an ester to the phosphate group, of the general formula

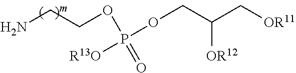

wherein R¹¹ and R¹² are each independently hydrogen or an acyl group, and R¹³ is a negative charge or a hydrogen, depending upon pH. When R13 is a negative charge, a suitable counterion, such as a sodium ion, can be present. For example, the alkanolamine moiety can be an ethanolamine moiety, such that m=1. It is also understood that the NH group can be protonated and positively charged, or unprotonated and neutral, depending upon pH. For example, the phospholipid can exist as a zwitterion with a negatively charged phosphate oxy anion and a positively charged protonated nitrogen atom. The carbon atom bearing OR¹² is a chiral carbon atom, so the molecule can exist as an R isomer, an S isomer, or any mixture thereof. When there are equal amounts of R and S isomers in a sample of the compound of formula (II), the sample is referred to as a “racemate.” For example in the commercially available product 1,2-dioleoyl-sn-glycero-3-phosphoethanolamine, as used in Example I below, the R³ group is of the chiral structure

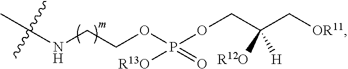

which is of the R absolute configuration.

A phospholipid can be either a free molecule, or covalently linked to another group for example as shown

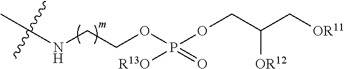

wherein a wavy line indicates a point of bonding.

Accordingly, when a substituent group, such as R³ of the compound of formula (I) herein, is stated to be a phospholipid what is meant that a phospholipid group is bonded as specified by the structure to another group, such as to an N-benzyl heterocyclic ring system as disclosed herein. The point of attachment of the phospholipid group can be at any chemically feasible position unless specified otherwise, such as by a structural depiction. For example, in the phospholipid structure shown above, the point of attachment to another chemical moiety can be via the ethanolamine nitrogen atom, for example as an amide group by bonding to a carbonyl group of the other chemical moiety, for example

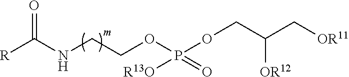

wherein R represents the other chemical moiety to which the phospholipid is bonded. In this bonded, amide derivative, the R¹³ group can be a proton or can be a negative charge associated with a counterion, such as a sodium ion. The acylated nitrogen atom of the alkanolamine group is no longer a basic amine, but a neutral amide, and as such is not protonated at physiological pH.

An “acyl” group as the term is used herein refers to an organic structure bearing a carbonyl group through which the structure is bonded, e.g., to glycerol hydroxyl groups of a phospholipid, forming a “carboxylic ester” group. Examples of acyl groups include fatty acid groups such as oleoyl groups, that thus form fatty (e.g., oleoyl) esters with the glycerol hydroxyl groups. Accordingly, when R¹¹ or R¹², but not both, are acyl groups, the phospholipid shown above is a mono-carboxylic ester, and when both R¹¹ and R¹² are acyl groups, the phospholipid shown above is a di-carboxylic ester. Within the present invention it is to be understood that a compound of the formula (I) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

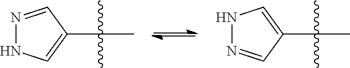 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

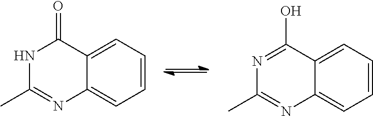

is an example of tautomerism. Accordingly, a structure depicted herein as one tautomer is intended to also include the other tautomer.

### Optical Isomerism

It will be understood that when compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

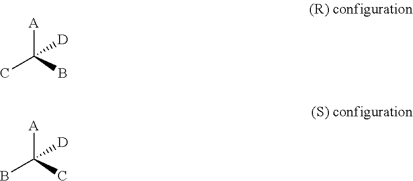

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. In one embodiment, the isolated isomer is at least about 80%, e.g., at least 90%, 98% or 99% pure, by weight.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, behenic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be employed. Lists of suitable salts are found in Remington's Pharmaceutical Sciences, 17th ed., Mack Publishing Company, Easton, Pa., p. 1418 (1985), the disclosure of which is hereby incorporated by reference.

The compounds of the formulas described herein can be solvates, and in some embodiments, hydrates. The term “solvate” refers to a solid compound that has one or more solvent molecules associated with its solid structure. Solvates can form when a compound is crystallized from a solvent. A solvate forms when one or more solvent molecules become an integral part of the solid crystalline matrix upon solidification. The compounds of the formulas described herein can be solvates, for example, ethanol solvates. Another type of a solvate is a hydrate. A “hydrate” likewise refers to a solid compound that has one or more water molecules intimately associated with its solid or crystalline structure at the molecular level. Hydrates can form when a compound is solidified or crystallized in water, where one or more water molecules become an integral part of the solid crystalline matrix.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Het can be heteroaryl, which encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine agonist activity using the standard tests described herein, or using other similar tests which are well known in the art. It is also understood by those of skill in the art that the compounds described herein include their various tautomers, which can exist in various states of equilibrium with each other.

The terms “treat” and “treating” as used herein refer to (i) preventing a pathologic condition from occurring (e.g., prophylaxis); (ii) inhibiting the pathologic condition or arresting its development; (iii) relieving the pathologic condition; and/or (iv) ameliorating, alleviating, lessening, and removing symptoms of a condition. A candidate molecule or compound described herein may be in an amount in a formulation or medicament, which is an amount that can lead to a biological effect, or lead to ameliorating, alleviating, lessening, relieving, diminishing or removing symptoms of a condition, e.g., disease, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism (microbe) in a system (e.g., cell, tissue, or subject) infected with a microbe, reducing the rate of microbial propagation, reducing the number of symptoms or an effect of a symptom associated with the microbial infection, and/or removing detectable amounts of the microbe from the system. Examples of microbe include but are not limited to virus, bacterium and fungus.

The term “therapeutically effective amount” as used herein refers to an amount of a compound, or an amount of a combination of compounds, to treat or prevent a disease or disorder, or to treat a symptom of the disease or disorder, in a subject. As used herein, the terms “subject” and “patient” generally refers to an individual who will receive or who has received treatment (e.g., administration of a compound) according to a method described herein.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

### TLR7 Agonists and Conjugates and Uses Thereof.

In various embodiments, the invention provides a method to prevent, inhibit or treat a microbial infection or a condition such as one associated with inflammation in a mammal. The methods include administering to a mammal in need thereof an effective amount of a composition comprising an amount of a compound of Formula (I):

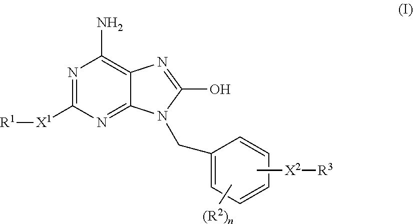

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R³ is a phospholipid comprising one or two carboxylic esters;

or a tautomer thereof;

or a pharmaceutically acceptable salt or solvate thereof. Optionally, the composition further comprises an antigen. In one embodiment, the composition having an antigen is administered concurrently, prior to or subsequent to administration of the composition having a compound of formula (I).

For example, R³ can comprise a group of formula

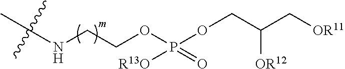

wherein R¹¹ and R¹² are each independently a hydrogen or an acyl group, R¹³ is a negative charge or a hydrogen, and m is 1 to 8, wherein a wavy line indicates a position of bonding, wherein an absolute configuration at the carbon atom bearing OR¹² is R, S, or any mixture thereof.

For example, m can be 1, providing a glycerophosphatidylethanolamine. More specifically, R¹¹ and R¹² can each be oleoyl groups.

In various embodiments, the phospholipid of R³ can comprise two carboxylic esters and each carboxylic ester includes one, two, three or four sites of unsaturation, epoxidation, hydroxylation, or a combination thereof.

In various embodiments, the phospholipid of R³ can comprise two carboxylic esters and the carboxylic esters of are the same or different. More specifically, each carboxylic ester of the phospholipid can be a C17 carboxylic ester with a site of unsaturation at C8-C9. Alternatively, each carboxylic ester of the phospholipid can be a C18 carboxylic ester with a site of unsaturation at C9-C10.

In various embodiments, X² can be a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups. The chain can be interspersed with one or more cycloalkyl, aryl, heterocyclyl, or heteroaryl rings.

In various embodiments, X² can be C(O), or can be any of

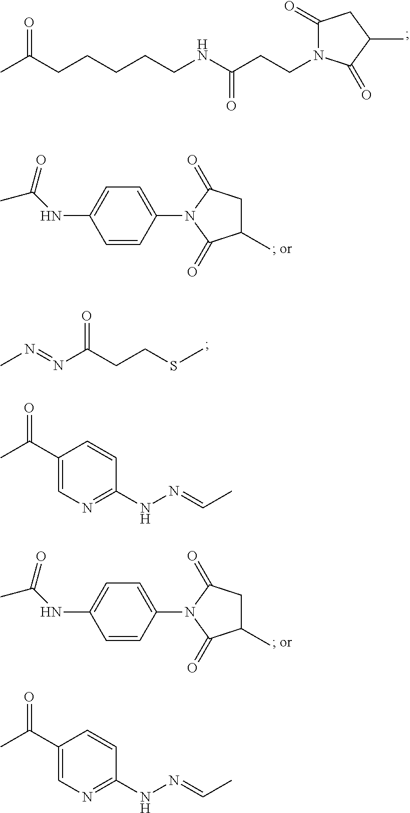

In various embodiments, R³ can be dioleoylphosphatidyl ethanolamine (DOPE).

In various embodiments, R³ can be 1,2-dioleoyl-sn-glycero-3-phospho ethanolamine and X² can be C(O).

In various embodiments, X¹ can be oxygen.

In various embodiments, X¹ can be sulfur, or can be —NR^{c}— where R^{c} is hydrogen, C₁₋₆ alkyl or substituted C₁₋₆ alkyl, where the alkyl substituents are hydroxy, C₃₋₆cycloalkyl, C₁₋₆alkoxy, amino, cyano, or aryl. More specifically, X¹ can be —NH—.

In various embodiments, R¹ and R^{c} taken together can form a heterocyclic ring or a substituted heterocyclic ring. More specifically, R¹ and R^{c} taken together can form a substituted or unsubstituted morpholino, piperidino, pyrrolidino, or piperazino ring.

In various embodiments R¹ can be a C1-C10 alkyl substituted with C1-6 alkoxy.

In various embodiments, R¹ can be hydrogen, C₁₋₄alkyl, or substituted C₁₋₄alkyl. More specifically, R¹ can be hydrogen, methyl, ethyl, propyl, butyl, hydroxyC₁₋₄alkylene, or C₁₋₄alkoxyC₁₋₄alkylene. Even more specifically, R¹ can be hydrogen, methyl, ethyl, methoxyethyl, or ethoxyethyl.

In various embodiments, R² can be absent, or R² can be halogen or C₁₋₄alkyl. More specifically, R² can be chloro, bromo, methyl, or ethyl.

In various embodiments, X¹ can be O, R¹ can be C₁₋₄alkoxy-ethyl, n can be 1, R² can be hydrogen, X² can be carbonyl, and R³ can be 1,2-dioleoylphosphatidyl ethanolamine (DOPE).

In various embodiments, the compound of Formula (I) can be:

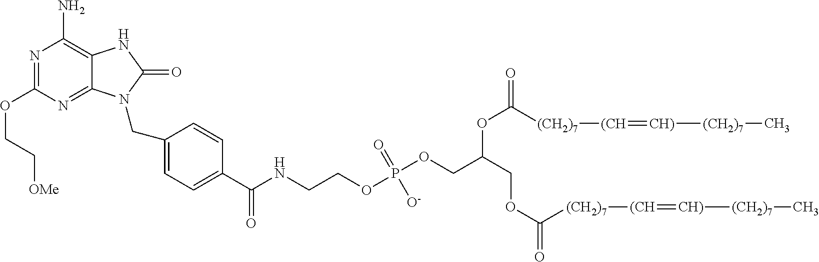

In various embodiments, the compound of formula (I) can be the R-enantiomer of the above structure:

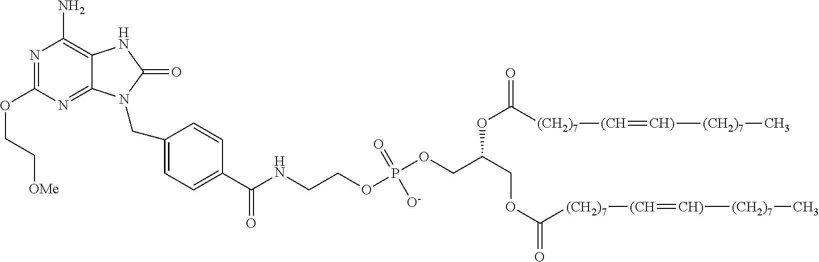

In various embodiments, the microbe is a bacteria, or, the antigen can comprise bacterial spores.

In various embodiments, the amount is effective to prevent infection.

In various embodiments, the mammal can be a human.

In various embodiments, the composition can be intranasally administered, or can be dermally administered.

In various embodiments, a phospholipid conjugate such as IV270 can be can be incorporated into a nanoparticle such as those described in WO 2010/083337, the disclosure of which is incorporated by reference herein.

In various embodiments, a phospholipid conjugate such as IV270 can be prepared in the form of a nanoparticulate suspension of the phospholipid conjugate in combination with a lipid and/or a phospholipid in an aqueous medium (e.g., a nanoliposome). A nanoliposome is a submicron bilayer lipid vesicle (see Chapter 2 by Mozafari in: Liposomes, Methods in Molecular Biology, vol. 605, V. Weissing (ed.), Humana Press, the disclosure of which is incorporated by reference herein). Nanoliposomes provide more surface area and may increase solubility, bioavailability and targeting.

Lipids are fatty acid derivatives with various head group moieties. Triglycerides are lipids made from three fatty acids and a glycerol molecule (a three-carbon alcohol with a hydroxyl group [OH] on each carbon atom). Mono- and diglycerides are glyceryl mono- and di-esters of fatty acids. Phospholipids are similar to triglycerides except that the first hydroxyl of the glycerol molecule has a polar phosphate-containing group in place of the fatty acid. Phospholipids are amphiphilic, possessing both hydrophilic (water soluble) and hydrophobic (lipid soluble) groups. The head group of a phospholipid is hydrophilic and its fatty acid tail (acyl chain) is hydrophobic. The phosphate moiety of the head group is negatively charged.

In addition to lipid and/or phospholipid molecules, nanoliposomes may contain other molecules such as sterols in their structure. Sterols are important components of most natural membranes, and incorporation of sterols into nanoliposome bilayers can bring about major changes in the properties of these vesicles. The most widely used sterol in the manufacture of the lipid vesicles is cholesterol (Chol). Cholesterol does not by itself form bilayer structures; but it can be incorporated into phospholipid membranes in very high concentrations, for example up to 1:1 or even 2:1 molar ratios of cholesterol to a phospholipid such as phosphatidylcholine (PC) (11). Cholesterol is used in nanoliposome structures in order to increase the stability of the vesicles by modulating the fluidity of the lipid bilayer. In general, cholesterol modulates fluidity of phospholipid membranes by preventing crystallization of the acyl chains of phospholipids and providing steric hindrance to their movement. This contributes to the stability of nanoliposomes and reduces the permeability of the lipid membrane to solutes.

Physicochemical properties of nanoliposomes depend on several factors including pH, ionic strength and temperature. Generally, lipid vesicles show low permeability to the entrapped material. However, at elevated temperatures, they undergo a phase transition that alters their permeability. Phospholipid ingredients of nanoliposomes have an important thermal characteristic, i.e., they can undergo a phase transition (Tc) at temperatures lower than their final melting point (Tm). Also known as gel to liquid crystalline transition temperature, Tc is a temperature at which the lipidic bilayer loses much of its ordered packing while its fluidity increases. Phase transition temperature of phospholipid compounds and lipid bilayers depends on the following parameters: polar head group; acyl chain length; degree of saturation of the hydrocarbon chains; and nature and ionic strength of the suspension medium. In general, Tc is lowered by decreased chain length, by unsaturation of the acyl chains, as well as presence of branched chains and bulky head groups (e.g. cyclopropane rings).

Hydrated phospholipid molecules arrange themselves in the form of bilayer structures via Van-der Waals and hydrophilic/hydrophobic interactions. In this process, the hydrophilic head groups of the phospholipid molecules face the water phase while the hydrophobic region of each of the monolayers face each other in the middle of the membrane. It should be noted that formation of liposomes and nanoliposomes is not a spontaneous process and sufficient energy must be put into the system to overcome an energy barrier. In other words, lipid vesicles are formed when phospholipids such as lecithin are placed in water and consequently form bilayer structures, once adequate amount of energy is supplied. Input of energy (e.g. in the form of sonication, homogenisation, heating, etc.) results in the arrangement of the lipid molecules, in the form of bilayer vesicles, to achieve a thermodynamic equilibrium in the aqueous phase.

For example, a composition comprising a compound of the invention such as IV270 as a mixture with a lipid such as cholesterol or a phospholipid such as phosphatidylcholine can be dispersed into a nanoparticulate form wherein lipid or phospholipid nanoparticles contain the TLR7 ligand conjugate associated therewith. By a nanoparticulate composition is meant a composition comprising nanoparticles, and a nanoparticle as the term is used herein refers to particles of about 1-1000 nm is diameter. As discussed below in Example IV, a nanoparticulate/nanoliposome composition is prepared using IV270 and the phophatidylcholine preparation Phosal 50 PG®.

In one embodiment, the invention provides a prophylactic or therapeutic method for preventing or treating a pathological condition or symptom in a mammal, such as a human, wherein the activity of a TLR7 agonist is implicated and its action is desired. The method includes administering to a mammal in need of such therapy, an antigen and an effective amount of a conjugate of the invention, or a pharmaceutically acceptable salt thereof. Non-limiting examples of pathological conditions or symptoms that are suitable for treatment include cancers, microbial infections or diseases, e.g., skin or bladder diseases. In one embodiment, the conjugates of the invention can be used to prepare vaccines against bacteria, viruses, cancer cells, or cancer-specific peptides, as a CNS stimulant, or for biodefense. The invention thus provides a conjugate for use alone or with other therapeutic agents in medical therapy (e.g., for use as an anti-cancer agent, to prevent, inhibit or treat bacterial diseases, to prevent, inhibit or treat viral diseases, such as hepatitis C and hepatitis B, and generally as agents for enhancing the immune response).

In one embodiment, the present invention provides a method for preventing, inhibiting or treating cancer by administering an effective amount of a cancer antigen and a TLR7 agonist phospholipid conjugate of the invention. The cancer may be an interferon sensitive cancer, such as, for example, a leukemia, a lymphoma, a myeloma, a melanoma, or a renal cancer. Specific cancers that can be treated include melanoma, superficial bladder cancer, actinic keratoses, intraepithelial neoplasia, and basal cell skin carcinoma, squamous, and the like. In addition, the method of the invention includes treatment for a precancerous condition such as, for example, actinic keratoses or intraepithelial neoplasia, familial polyposis (polyps), cervical dysplasia, cervical cancers, superficial bladder cancer, and any other cancers associated with infection (e.g., lymphoma Karposi's sarcoma, or leukemia); and the like.

In one embodiment, the invention provides a method to prevent or inhibit a gram-positive bacterial infection in a mammal, comprising administering to the mammal an effective amount of a composition comprising a bacterial antigen of a gram-positive bacteria and an amount of a synthetic TLR7 agonist phospholipid conjugate. In one embodiment, a synthetic TLR7 agonist phospholipid conjugate is administered with one or more antigens of B. anthracis. In one embodiment, a synthetic TLR7 agonist phospholipid conjugate is administered with one or more antigens of S. aureus. Table 1 provides exemplary antigens for S. aureus. The vaccines of the invention may unexpectedly provide a rapid and effective immune response.

TABLE 1
Staphylococcus aureus immunogens

Weapon
Exfoliative toxin B
Exfoliative toxin A
Toxic shock-syndrome toxin
Enterotoxin A-E, H-U
Bone sialoprotein-binding protein
Collagen-binding protein
Clumping factor A
Clumping factor B
α-hemolysin
γ-hemolysin
Protein A
Clumping factor A
Fibronectin-binding protein A
Fibronectin-binding protein B
Collagen-binding protein
Lipoteichoic acid
Peptidoglycan
Protein A
Fibronectin-binding protein B
α-hemolysin
Panton valentine leukocidin
Collagen-binding protein
Lipoteichoic acid
Peptidoglycan
Capsular polysaccharide
Clumping factor A
Protein A
Fibronectin-binding proteins

Other disorders that may be amenable to treatment that includes a TLR7 agonist phospholipid conjugate of the invention or a pharmaceutically acceptable salt of such a compound include, but are not limited to Multiple Sclerosis, lupus, rheumatoid arthritis, Crohn's Disease and the like.

The TLR agonist conjugates may include a homofunctional TLR agonist, e.g., formed of a TLR7 agonist. The TLR7 agonist can be a 7-thia-8-oxoguanosinyl (TOG) moiety, a 7-deazaguanosinyl (7DG) moiety, a resiquimod moiety, or an imiquimod moiety. In another embodiment, the TLR agonist conjugate may include a heterofunctional TLR agonist polymer. The heterofunctional TLR agonist polymer may include a TLR7 agonist and a TLR3 agonist or a TLR9 agonist, or all three agonists.

In one embodiment, the invention provides the following conjugates

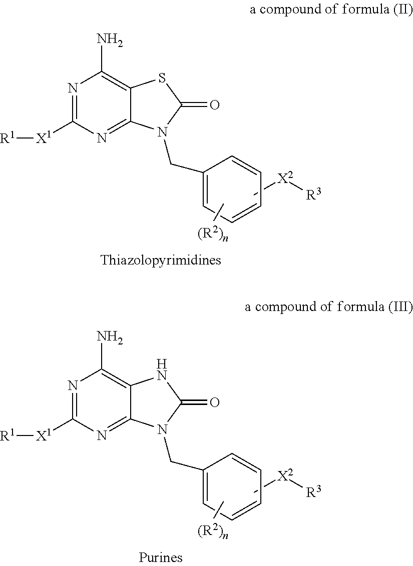

X¹=—O—, —S—, or —NR^{C}—,

wherein R^{c} hydrogen, C₁₋₁₀alkyl, or C₁₋₁₀alkyl substituted by C₃₋₆ cycloalkyl, or R^{c} and R¹ taken together with the nitrogen atom can form a heterocyclic ring or a substituted heterocyclic ring, wherein the substituents are hydroxy, C₁₋₆ alkyl, hydroxy C₁₋₆ alkylene, C₁₋₆ alkoxy, C₁₋₆ alkoxy C₁₋₆ alkylene, or cyano;

wherein R¹ is (C₁-C₁₀)alkyl, substituted (C₁-C₁₀)alkyl, C₆₋₁₀ aryl, or substituted C₆₋₁₀ aryl, C₅₋₉ heterocyclic, substituted C₅₋₉ heterocyclic; wherein the substituents on the alkyl, aryl or heterocyclic groups are hydroxy, C₁₋₆ alkyl, hydroxy C₁₋₆ alkylene, C₁₋₆ alkoxy, C₁₋₆ alkoxy C₁₋₆ alkylene, amino, cyano, halogen, or aryl;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), —O—C(O)NR^{a}R^{b}, —(C₁-C₆)alkylene-NR^{a}R^{b}, —(C₁-C₆)alkylene-C(O)NR^{a}R^{b}, halo, nitro, or cyano;

wherein each R^{a} and R^{b} is independently hydrogen, (C₁₋₆)alkyl, (C₃-C₈)cycloalkyl, (C₁₋₆6)alkoxy, halo(C₁₋₆)alkyl, (C₃-C₈)cycloalkyl(C₁₋₆)alkyl, (C_{I}-6)alkanoyl, hydroxy(C₁₋₆)alkyl, aryl, aryl(C₁₋₆)alkyl, aryl, aryl(C₁₋₆)alkyl, Het, Het (C₁₋₆)alkyl, or (C₁₋₆)alkoxycarbonyl; wherein X² is a bond or a linking group; wherein R³ is a phospholipid comprising one or two carboxylic esters wherein n is 0, 1, 2, 3, or 4; or a tautomer thereof; or a pharmaceutically acceptable salt thereof.

In cases where compounds are sufficiently basic or acidic to form acid or base salts, use of the compounds as salts may be appropriate. Examples of acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Alkyl includes straight or branched C₁₋₁₀ alkyl groups, e.g., methyl, ethyl, propyl, butyl, pentyl, isopropyl, isobutyl, 1-methylpropyl, 3-methylbutyl, hexyl, and the like.

Lower alkyl includes straight or branched C₁₋₆ alkyl groups, e.g., methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, and the like.

The term “alkylene” refers to a divalent straight or branched hydrocarbon chain (e.g., ethylene: —CH₂—CH₂—).

C₃₋₇ Cycloalkyl includes groups such as, cyclopropyl, cyclopentyl, cyclohexyl, cycloheptyl, and the like, and alkyl-substituted C₃₋₇ cycloalkyl group, preferably straight or branched C₁₋₆ alkyl group such as methyl, ethyl, propyl, butyl or pentyl, and C₅₋₇ cycloalkyl group such as, cyclopentyl or cyclohexyl, and the like.

Lower alkoxy includes C₁₋₆ alkoxy groups, such as methoxy, ethoxy or propoxy, and the like.

Lower alkanoyl includes C₁₋₆ alkanoyl groups, such as formyl, acetyl, propanoyl, butanoyl, pentanoyl or hexanoyl, and the like.

C₇₋₁₁ aroyl, includes groups such as benzoyl or naphthoyl;

Lower alkoxycarbonyl includes C₂₋₇ alkoxycarbonyl groups, such as methoxycarbonyl, ethoxycarbonyl or propoxycarbonyl, and the like.

Lower alkylamino group means amino group substituted by C₁₋₆ alkyl group, such as, methylamino, ethylamino, propylamino, butylamino, and the like.

Di(lower alkyl)amino group means amino group substituted by the same or different and C₁₋₆ alkyl group (e.g., dimethylamino, diethylamino, ethylmethylamino).

Lower alkylcarbamoyl group means carbamoyl group substituted by C₁₋₆ alkyl group (e.g., methylcarbamoyl, ethylcarbamoyl, propylcarbamoyl, butylcarbamoyl).

Di(lower alkyl)carbamoyl group means carbamoyl group substituted by the same or different and C₁₋₆ alkyl group (e.g., dimethylcarbamoyl, diethylcarbamoyl, ethylmethylcarbamoyl).

Halogen atom means halogen atom such as fluorine atom, chlorine atom, bromine atom or iodine atom.

Aryl refers to a C₆₋₁₀ monocyclic or fused cyclic aryl group, such as phenyl, indenyl, or naphthyl, and the like.

Heterocyclic or heterocycle refers to monocyclic saturated heterocyclic groups, or unsaturated monocyclic or fused heterocyclic group containing at least one heteroatom, e.g., 0-3 nitrogen atoms NR^{c}, 0-1 oxygen atom (—O—), and 0-1 sulfur atom (—S—). Non-limiting examples of saturated monocyclic heterocyclic group includes 5 or 6 membered saturated heterocyclic group, such as tetrahydrofuranyl, pyrrolidinyl, morpholinyl, piperidyl, piperazinyl or pyrazolidinyl. Non-limiting examples of unsaturated monocyclic heterocyclic group includes 5 or 6 membered unsaturated heterocyclic group, such as furyl, pyrrolyl, pyrazolyl, imidazolyl, thiazolyl, thienyl, pyridyl or pyrimidinyl. Non-limiting examples of unsaturated fused heterocyclic groups includes unsaturated bicyclic heterocyclic group, such as indolyl, isoindolyl, quinolyl, benzothizolyl, chromanyl, benzofuranyl, and the like. A Het group can be a saturated heterocyclic group or an unsaturated heterocyclic group, such as a heteroaryl group.

R^{c} and R¹ taken together with the nitrogen atom to which they are attached can form a heterocyclic ring. Non-limiting examples of heterocyclic rings include 5 or 6 membered saturated heterocyclic rings, such as 1-pyrrolidinyl, 4-morpholinyl, 1-piperidyl, 1-piperazinyl or 1-pyrazolidinyl, 5 or 6 membered unsaturated heterocyclic rings such as 1-imidazolyl, and the like.

The alkyl, aryl, heterocyclic groups of R¹ can be optionally substituted with one or more substituents, wherein the substituents are the same or different, and include lower alkyl; cycloalkyl, hydroxyl; hydroxy C₁₋₆ alkylene, such as hydroxymethyl, 2-hydroxyethyl or 3-hydroxypropyl; lower alkoxy; C₁₋₆ alkoxy C₁₋₆ alkyl, such as 2-methoxyethyl, 2-ethoxyethyl or 3-methoxypropyl; amino; alkylamino; dialkyl amino; cyano; nitro; acyl; carboxyl; lower alkoxycarbonyl; halogen; mercapto; C₁₋₆ alkylthio, such as, methylthio, ethylthio, propylthio or butylthio; substituted C₁₋₆ alkylthio, such as methoxyethylthio, methylthioethylthio, hydroxyethylthio or chloroethylthio; aryl; substituted C₆₋₁₀ monocyclic or fused-cyclic aryl, such as 4-hydroxyphenyl, 4-methoxyphenyl, 4-fluorophenyl, 4-chlorophenyl or 3,4-dichlorophenyl; 5-6 membered unsaturated heterocyclic, such as furyl, pyrrolyl, pyrazolyl, imidazolyl, thiazolyl, thienyl, pyridyl or pyrimidinyl; and bicyclic unsaturated heterocyclic, such as indolyl, isoindolyl, quinolyl, benzothiazolyl, chromanyl, benzofuranyl or phthalimino. In certain embodiments, one or more of the above groups can be expressly excluded as a substituent of various other groups of the formulas.

The alkyl, aryl, heterocyclic groups of R² can be optionally substituted with one or more substituents, wherein the substituents are the same or different, and include hydroxyl; C₁₋₆ alkoxy, such as methoxy, ethoxy or propoxy; carboxyl; C₂₋₇ alkoxycarbonyl, such as methoxycarbonyl, ethoxycarbonyl or propoxycarbonyl) and halogen.

The alkyl, aryl, heterocyclic groups of R^{c} can be optionally substituted with one or more substituents, wherein the substituents are the same or different, and include C₃₋₆ cycloalkyl; hydroxyl; C₁₋₆ alkoxy; amino; cyano; aryl; substituted aryl, such as 4-hydroxyphenyl, 4-methoxyphenyl, 4-chlorophenyl or 3,4-dichlorophenyl; nitro and halogen.

The heterocyclic ring formed together with R^{c} and R¹ and the nitrogen atom to which they are attached can be optionally substituted with one or more substituents, wherein the substituents are the same or different, and include C₁₋₆ alkyl; hydroxy C₁₋₆ alkylene; C₁₋₆ alkoxy C₁₋₆ alkylene; hydroxyl; C₁₋₆ alkoxy; and cyano. A specific value for X¹ is a sulfur atom, an oxygen atom or —NR^{S}—. Another specific X¹ is a sulfur atom.

Another specific X¹ is an oxygen atom.

Another specific X¹ is —NR^{c}—.

Another specific X¹ is —NH—.

A specific value for R^{c} is hydrogen, C₁ alkyl or substituted C₁₋₄ alkyl.

A specific value for R¹ and R^{c} taken together is when they form a heterocyclic ring or a substituted heterocyclic ring.

Another specific value for R¹ and R^{c} taken together is substituted or unsubstituted morpholino, piperidino, pyrrolidino, or piperazino ring

A specific value for R¹ is hydrogen, C₁₋₁₀alkyl, or substituted C₁₋₄alkyl.

Another specific R¹ is 2-hydroxyethyl, 3-hydroxypropyl, 4-hydroxybutyl, 2-aminoethyl, 3-aminopropyl, 4-aminobutyl, methoxymethyl, 2-methoxyethyl, 3-methoxypropyl, ethoxymethyl, 2-ethoxyethyl, methylthiomethyl, 2-methylthioethyl, 3-methylthiopropyl, 2-fluoroethyl, 3-fluoropropyl, 2,2,2-trifluoroethyl, cyanomethyl, 2-cyanoethyl, 3-cyanopropyl, methoxycarbonylmethyl, 2-methoxycarbonylethyl, 3-methoxycarbonylpropyl, benzyl, phenethyl, 4-pyridylmethyl, cyclohexylmethyl, 2-thienylmethyl, 4-methoxyphenylmethyl, 4-hydroxyphenylmethyl, 4-fluorophenylmethyl, or 4-chlorophenylmethyl.

Another specific R¹ is hydrogen, CH₃—, CH₃—CH₂—, CH₃CH₂CH₂—, hydroxyC₁₋₄alkylene, or C₁₋₄alkoxyC₁₋₄alkylene.

Another specific value for R¹ is hydrogen, CH₃—, CH₃—CH₂—, CH₃—O—CH₂CH₂— or CH₃—CH₂—O—CH₂CH₂—.

A specific value for R² is halogen or C₁₋₄alkyl.

Another specific value for R² is chloro, bromo, CH₃—, or CH₃—CH₂—.

Specific substituents for substitution on the alkyl, aryl or heterocyclic groups are hydroxy, C₁₋₆alkyl, hydroxyC₁₋₆alkylene, C₁₋₆alkoxy, C₁₋₆alkoxyC₁-6alkylene, C₃₋₆cycloalkyl, amino, cyano, halogen, or aryl.

A specific value for X² is a bond or a chain having up to about 24 atoms; wherein the atoms are selected from the group consisting of carbon, nitrogen, sulfur, non-peroxide oxygen, and phosphorous. Any carbon atom can bear an oxo group, and any sulfur atom can bear one or two oxo groups. The chain can be interspersed with one or more cycloalkyl, aryl, heterocyclyl, or heteroaryl rings.

Another specific value for X² is a bond or a chain having from about 4 to about 12 atoms.

Another specific value for X² is a bond or a chain having from about 6 to about 9 atoms.

Another specific value for X² is a carbonyl (C(O)) group.

Certain non-limiting examples of X² include —(Y)_{y}—, —(Y)_{y}—C(O)N—(Z)_{z}—, —(CH₂)_{y}—C(O)N—(CH₂)_{z}—, —(Y)_{y}—NC(O)—(Z)_{z}—, —(CH₂)_{y}—NC(O)—(CH₂)_{z}—, where each y (subscript) and z (subscript) independently is 0 to 20 and each Y and Z independently is C1-C10 alkyl, substituted C1-C10 alkyl, C1-C10 alkoxy, substituted C1-C10 alkoxy, C3-C9 cycloalkyl, substituted C3-C9 cycloalkyl, C5-C10 aryl, substituted C5-C10 aryl, C5-C9 heterocyclic, substituted C5-C9 heterocyclic, C1-C6 alkanoyl, Het, Het C1-C6 alkyl, or C1-C6 alkoxycarbonyl, wherein the substituents on the alkyl, cycloalkyl, alkanoyl, alkcoxycarbonyl, Het, aryl or heterocyclic groups are hydroxyl, C1-C10 alkyl, hydroxyl C1-C10 alkylene, C1-C6 alkoxy, C3-C9 cycloalkyl, C5-C9 heterocyclic, C1-6 alkoxy C1-6 alkenyl, amino, cyano, halogen or aryl. In certain embodiments, a linker sometimes is a —C(Y′)(Z′)—C(Y″)(Z″)— linker, where each Y′, Y″, Z′ and Z″ independently is hydrogen C1-C10 alkyl, substituted C1-C10 alkyl, C1-C10 alkoxy, substituted C1-C10 alkoxy, C3-C9 cycloalkyl, substituted C3-C9 cycloalkyl, C5-C10 aryl, substituted C5-C10 aryl, C5-C9 heterocyclic, substituted C5-C9 heterocyclic, C1-C6 alkanoyl, Het, Het C1-C6 alkyl, or C1-C6 alkoxycarbonyl, wherein the substituents on the alkyl, cycloalkyl, alkanoyl, alkcoxycarbonyl, Het, aryl or heterocyclic groups are hydroxyl, C1-C10 alkyl, hydroxyl C1-C10 alkylene, C1-C6 alkoxy, C3-C9 cycloalkyl, C5-C9 heterocyclic, C1-6 alkoxy C1-6 alkenyl, amino, cyano, halogen or aryl.

Another specific value for X² is

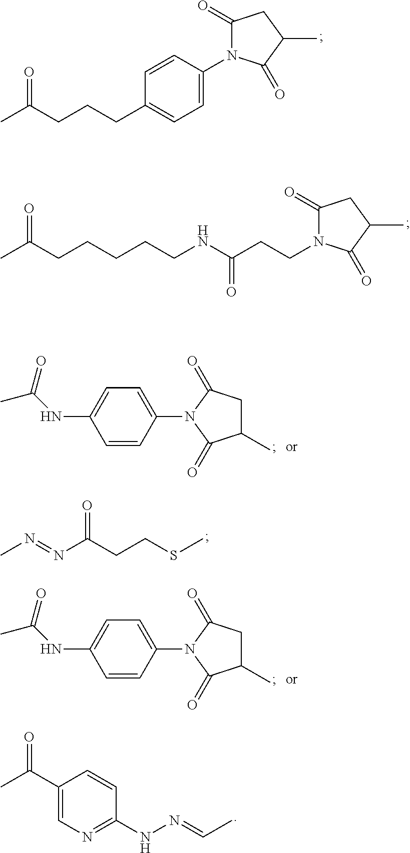

Another specific value for X² is

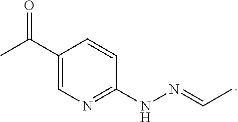

A specific antigen includes an amino acid, a carbohydrate, a peptide, a protein, a nucleic acid, a lipid, a body substance, or a cell such as a microbe.

A specific peptide has from 2 to about 20 amino acid residues.

Another specific peptide has from 10 to about 20 amino acid residues.

A specific antigen includes a carbohydrate.

A specific antigen is a microbe. A specific microbe is a virus, bacteria, or fungi.

Specific bacteria are Bacillus anthracis, Listeria monocytogenes, Francisella tularensis, Salmonella, or Staphylococcus. Specific Salmonella are S. typhimurium or S. enteritidis. Specific Staphylococcus include S. aureus.

Specific viruses are RNA viruses, including RSV and influenza virus, a product of the RNA virus, or a DNA virus, including herpes virus. A specific DNA virus is hepatitis B virus.

The invention includes compositions that include of a TLR7 agonist phospholipid conjugate of the invention optionally in combination with other active agents that may or may not be antigens, e.g., ribavirin, mizoribine, and mycophenolate mofetil. Other non-limiting examples are known and are disclosed in U.S. published patent application No. 20050004144.

Processes for preparing intermediates useful for preparing compounds of the invention and formulations having one or more of those compounds, are provided as further embodiments of the invention. Intermediates useful for preparing compounds of the invention are also provided as further embodiments of the invention.

Administration of compositions having conjugates of the invention, e.g., administration of a composition having a phospholipid conjugate of the invention and another active agent or administration of a composition having a phospholipid conjugate of the invention and a composition having another active agent, can be via any of suitable route of administration, particularly parenterally, for example, intravenously, intra-arterially, intraperitoneally, intrathecally, intraventricularly, intraurethrally, intrasternally, intracranially, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds (a conjugate or other active agent) may be formulated as a sterile solution in water or another suitable solvent or mixture of solvents. The solution may contain other substances such as salts, sugars (particularly glucose or mannitol), to make the solution isotonic with blood, buffering agents such as acetic, critic, and/or phosphoric acids and their sodium salts, and preservatives.

The phospholipid conjugates of the invention alone or in combination with other active agents can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

Thus, the present phospholipid conjugates alone or in combination with another active agent, e.g., an antigen, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the conjugate optionally in combination with an active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of conjugate and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the phospholipid conjugate optionally in combination with another active compound may be incorporated into sustained-release preparations and devices.

The phospholipid conjugate optionally in combination with another active compound may also be administered intravenously or intraperitoneally by infusion or injection. Solutions of the phospholipid conjugate optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating compound(s) in the required amount in the appropriate solvent with various of the other ingredients enumerated above, as required, followed by filter sterilization. In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the phospholipid conjugate optionally in combination with another active compound may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the invention provides various dosage formulations of the phospholipid conjugate optionally in combination with another active compound for inhalation delivery. For example, formulations may be designed for aerosol use in devices such as metered-dose inhalers, dry powder inhalers and nebulizers.

Examples of useful dermatological compositions which can be used to deliver compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages can be determined by comparing their in vitro activity, and in vivo activity in animal models. Methods for the extrapolation of effective dosages in mice, and other animals, to humans are known to the art; for example, see U.S. Pat. No. 4,938,949. The ability of a compound of the invention to act as a TLR agonist may be determined using pharmacological models which are well known to the art, including the procedures disclosed by Lee et al., Proc. Natl. Acad. Sci. USA, 100: 6646 (2003).

Generally, the concentration of the phospholipid conjugate optionally in combination with another active compound in a liquid composition, such as a lotion, will be from about 0.1-25 wt-%, e.g., from about 0.5-10 wt-%. The concentration in a semi-solid or solid composition such as a gel or a powder will be about 0.1-5 wt-%, e.g., about 0.5-2.5 wt-%.

The active ingredient may be administered to achieve peak plasma concentrations of the active compound of from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the phospholipid conjugate optionally in combination with another active compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day.

The phospholipid conjugate optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual patient. In general, the total daily dose range for an active agent for the conditions described herein, may be from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered compound. This can achieve plasma levels of about 500-750 uM, which can be effective to kill cancer cells. In managing the patient, the therapy should be initiated at a lower dose and increased depending on the patient's global response.

As described above, compositions that contain a phospholipid conjugate in combination with another active compound are useful in the treatment or prevention of a disease or disorder in, for example, humans or other mammals (e.g., bovine, canine, equine, feline, ovine, and porcine animals), and perhaps other animals as well. Depending on the particular compound, the composition will, for example, be useful for treating cancer, an infection, enhancing adaptive immunity (e.g., antibody production, T cell activation, etc.), as vaccines, and/or stimulating the central nervous system.

A phospholipid conjugate in conjunction with an antigen can be administered to a subject in need thereof to treat one or more inflammation disorders. As used hereinafter, the terms “treating,” “treatment” and “therapeutic effect” can refer to reducing, inhibiting or stopping (preventing) an inflammation response (e.g., slowing or halting antibody production or amount of antibodies to a specific antigen), reducing the amount of inflamed tissue and alleviating, completely or in part, an inflammation condition. Inflammation conditions include, without limitation, allergy, asthma, autoimmune disorder, chronic inflammation, chronic prostatitis, glomerulonephritis, hypersensitivities, inflammatory bowel diseases, myopathy (e.g., in combination with systemic sclerosis, dermatomyositis, polymyositis, and/or inclusion body myositis), pelvic inflammatory disease, reperfusion injury, rheumatoid arthritis, transplant rejection, vasculitis, and leukocyte disorders (e.g., Chediak-Higashi syndrome, chronic granulomatous disease). Certain autoimmune disorders also are inflammation disorders (e.g., rheumatoid arthritis). In some embodiments, the inflammation disorder is selected from the group consisting of chronic inflammation, chronic prostatitis, glomerulonephritis, a hypersensitivity, myopathy, pelvic inflammatory disease, reperfusion injury, transplant rejection, vasculitis, and leukocyte disorder. In certain embodiments, an inflammation condition includes, but is not limited to, bronchiectasis, bronchiolitis, cystic fibrosis, acute lung injury, acute respiratory distress syndrome (ARDS), atherosclerosis, and septic shock (e.g., septicemia with multiple organ failure). In some embodiments, an inflammation condition is not a condition selected from the group consisting of allergy, asthma, ARDS and autoimmune disorder. In certain embodiments, an inflammation condition is not a condition selected from the group consisting of gastrointestinal tract inflammation, brain inflammation, skin inflammation and joint inflammation. In certain embodiments, the inflammation condition is a neutrophil-mediated disorder.

A compound described herein can be administered to a subject in need thereof to potentially treat one or more autoimmune disorders. In such treatments, the terms “treating,” “treatment” and “therapeutic effect” can refer to reducing, inhibiting or stopping an autoimmune response (e.g., slowing or halting antibody production or amount of antibodies to a specific antigen), reducing the amount of inflamed tissue and alleviating, completely or in part, an autoimmune disorder. Autoimmune disorders, include, without limitation, autoimmune encephalomyelitis, colitis, automimmune insulin dependent diabetes mellitus (IDDM), and Wegener granulomatosis and Takayasu arteritis. Models for testing compounds for such diseases include, without limitation, (a)(i) C5BL/6 induced by myelin oligodendrocyte glycoprotein (MOG) peptide, (ii) SJL mice PLP139-151, or 178-191 EAE, and (iii) adoptive transfer model of EAE induced by MOG or PLP peptides for autoimmune encephalomyelitis; (b) non-obese diabetes (NOD) mice for autoimmune IDDM; (c) dextran sulfate sodium (DSS)-induced colitis model and trinitrobenzene sulfonic acid (TNBS)-induced colitis model for colitis; and (d) systemic small vasculitis disorder as a model for Wegener granulomatosis and Takayasu arteritis. A compound described herein may be administered to a subject to potentially treat one or more of the following disorders: Acute disseminated encephalomyelitis (ADEM); Addison's disease; alopecia greata; ankylosing spondylitis; antiphospholipid antibody syndrome (APS); autoimmune hemolytic anemia; autoimmune hepatitis; autoimmune inner ear disease; bullous pemphigoid; coeliac disease; Chagas disease; chronic obstructive pulmonary disease; Crohns disease (one of two types of idiopathic inflammatory bowel disease “IBD”); dermatomyositis; diabetes mellitus type 1; endometriosis; Goodpasture's syndrome; Graves' disease; Guillain-Barré syndrome (GBS); Hashimoto's disease; hidradenitis suppurativa; idiopathic thrombocytopenic purpura; interstitial cystitis; lupus erythematosus; mixed connective tissue disease; morphea; multiple sclerosis (MS); myasthenia gravis; narcolepsy; neuromyotonia; pemphigus vulgaris; pernicious anaemia; polymyositis; primary biliary cirrhosis; rheumatoid arthritis; schizophrenia; scleroderma; Sjögren's syndrome; temporal arteritis (also known as “giant cell arteritis”); ulcerative colitis (one of two types of idiopathic inflammatory bowel disease “IBD”); vasculitis; vitiligo; and Wegener's granulomatosis. In some embodiments, the autoimmune disorder or disease is not a disorder or disease selected from the group consisting of Chrohns disease (or Chrohn's disease), rheumatoid arthritis, lupus and multiple sclerosis.

A phospholipid conjugate and an antigen can be administered to a subject in need thereof to induce an immune response in the subject. The immune response may be generated automatically by the subject against a foreign antigen (e.g., pathogen infection) in certain embodiments. In some embodiments, an antigen is co-administered with a phospholipid conjugate described herein, where an immune response is mounted in the subject against the antigen. An antigen may be specific for a particular cell proliferative condition (e.g., a specific cancer antigen) or particular pathogen (e.g., gram positive bacteria wall antigen or S. aureus antigen), in certain embodiments. In some embodiments, a compound described herein induces little to no side effects (e.g., splenomegaly) when administered to a subject. In certain embodiments, a composition forms particles of about 10 nanometers to about 1000 nanometers, and sometimes, a composition forms particles with a mean, average or nominal size of about 100 nanometers to about 400 nanometers.

A phospholipid conjugate and an antigen can be administered to a subject in need thereof to potentially treat one or more cell proliferative disorders. In such treatments, the terms “treating,” “treatment” and “therapeutic effect” can refer to reducing or stopping a cell proliferation rate (e.g., slowing or halting tumor growth), reducing the number of proliferating cancer cells (e.g., ablating part or all of a tumor) and alleviating, completely or in part, a cell proliferation condition. Cell proliferative conditions include, but are not limited to, cancers of the colorectum, breast, lung, liver, pancreas, lymph node, colon, prostate, brain, head and neck, skin, liver, kidney, and heart. Examples of cancers include hematopoietic neoplastic disorders, which are diseases involving hyperplastic/neoplastic cells of hematopoietic origin (e.g., arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (reviewed in Vaickus, Crit. Rev. in Oncol./Hemotol. 11:267-297 (1991)); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. In a particular embodiment, the cell proliferative disorder is non-endocrine tumor or endocrine tumors. Illustrative examples of non-endocrine tumors include but are not limited to adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor may be an islet cell tumor.

Cell proliferative conditions also include inflammatory conditions, such as inflammation conditions of the skin, including, for example, eczema, discoid lupus erythematosus, lichen planus, lichen sclerosus, mycosis fungoides, photodermatoses, pityriasis rosea, psoriasis. Also included are cell proliferative conditions related to obesity, such as proliferation of adipocytes, for example.

Cell proliferative conditions also include viral diseases, including for example, acquired immunodeficiency syndrome, adenoviridae infections, alphavirus Infections, arbovirus Infections, Boma disease, bunyaviridae Infections, caliciviridae Infections, chickenpox, Ccoronaviridae Infections, coxsackievirus Infections, cytomegalovirus Infections, dengue, DNA Virus Infections, ecthyma, contagious, encephalitis, arbovirus, Epstein-Barr virus infections, erythema infectiosum, hantavirus infections, hemorrhagic fevers, viral, hepatitis, viral, human, herpes simplex, herpes zoster, herpes zoster oticus, herpesviridae infections, infectious mononucleosis, influenza, e.g., in birds or humans, Lassa fever, measles, Molluscum contagiosum, mumps, oaramyxoviridae Infections, phlebotomus fever, polyomavirus infections, rabies, respiratory syncytial virus Infections, Rift Valley fever, RNA Virus Infections, rubella, slow virus diseases, smallpox, subacute sclerosing panencephalitis, tumor virus infections, warts, West Nile fever, virus diseases and Yellow Fever. For example, Large T antigen of the SV40 transforming virus acts on UBF, activates it and recruits other viral proteins to Pol I complex, and thereby stimulates cell proliferation to ensure virus propagation. Cell proliferative conditions also include conditions related to angiogenesis (e.g., cancers) and obesity caused by proliferation of adipocytes and other fat cells.

Cell proliferative conditions also include cardiac conditions resulting from cardiac stress, such as hypertension, balloon angioplasty, valvular disease and myocardial infarction. For example, cardiomyocytes are differentiated muscle cells in the heart that constitute the bulk of the ventricle wall, and vascular smooth muscle cells line blood vessels. Although both are muscle cell types, cardiomyocytes and vascular smooth muscle cells vary in their mechanisms of contraction, growth and differentiation. Cardiomyocytes become terminally differentiated shortly after heart formation and thus loose the capacity to divide, whereas vascular smooth muscle cells are continually undergoing modulation from the contractile to proliferative phenotype. Under various pathophysiological stresses such as hypertension, baloon angioplasty, valvular disease and myocardial infarction, for example, the heart and vessels undergo morphologic growth-related alterations that can reduce cardiac function and eventually manifest in heart failure. Thus, provided herein are methods for treating cardiac cell proliferative conditions by administering a phospholipid conjugate and an antigen in an effective amount to treat the cardiac condition. The phospholipid conjugate and an antigen may be administered before or after a cardiac stress has occurred or has been detected, or administered after occurrence or detection of hypertension, balloon angioplasty, valvular disease or myocardial infarction, for example. Administration may decrease proliferation of vascular muscle cells and/or smooth muscle cells.

The invention will be further described by the following non-limiting examples.

### Example I

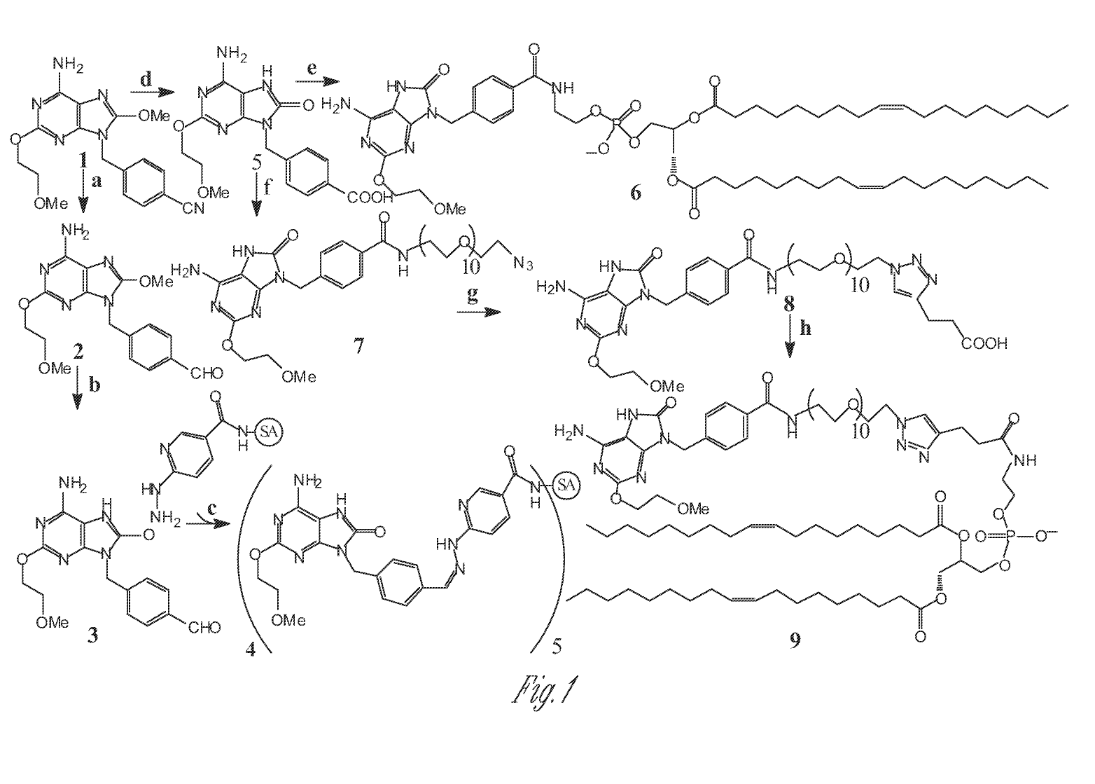

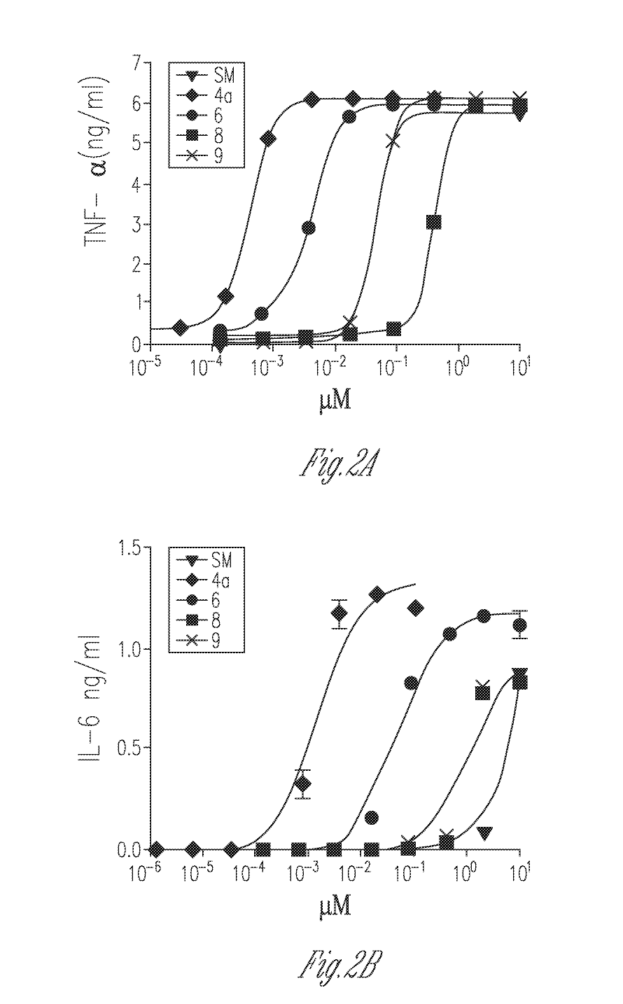

Chemical synthesis schemes described herein use numbers in parenthesis when referring to a compound in FIG. 1, and letters in parenthesis when referring to a reaction step (e.g., chemical(s) added and/or reaction conditions). For example, (a) refers to a reaction step that includes the addition of a reactant, which may result in the formation of compound (2), when combined and reacted with compound (1). The reaction conditions and compounds added for each reaction step are; (a) Lithium N,N′-methylethylenediaminoaluminum hydrides (Cha, J. et al., (2002)). Selective conversion of aromatic nitriles to aldehydes by lithium N,N′-dimethylethylenediaminoaluminum hydride, Bull. Korean Chem. Soc. 23, 1697-1698), THF, 0° C.; (b) NaI, chlorotrimethylsilane, CH₃CN, r.t.; (c) PBS, r.t.; (d) NaOH:EtOH 1:1, reflux; (e) DOPE, HATU, triethylamine, DMF/DCM 1:1, r.t.; (f) O-(2-Aminoethyl)-O′-(2-azidoethyl)nonaethylene glycol, HATU, triethylamine, DMF, r.t.; (g) 4 pentynoic acid, sodium ascorbate, Cu (OAc)₂, t-BuOH/H₂O/THF 2:2:1, r.t.; and (h) DOPE, HATU, triethylamine, DMF/DCM 1:1, r.t.

Synthesis of 4-((6-amino-2-(2-methoxyethoxy)-8-oxo-7H-purin-9(8H)-yl)methyl)benzoic acid (see FIG. 1, compound 5). 20 mL of a 1:1 ethanol:water mixture was added to 0.10 g (0.28 mmol) of 4-((6-amino-8-methoxy-2-(2-methoxyethoxy)-9H-purin-9 yl)methyl)benzonitrile (see FIG. 1, compound I), and the combination refluxed for 8 hours. The reaction mixture was allowed to cool and acidified to pH 2 with conc. HCl. The aqueous solution was further extracted with DCM (3×20 mL), dried over MgSO4 and evaporated in vacuo to yield a mixture of 8-oxo-9-benzoic acid (compound 5), 8-methoxy-9-benzoic acid and 8-oxo-9-ethyl benzoate. Once dried, the products were dissolved in CH₃CN (25 mL) and NaI (0.14 g, 0.96 mmol) was added (FIG. 1, reaction step (b)). To this solution was added 12 μL (0.96 mmol) of chlorotrimethylsilane, dropwise with stirring. The reaction mixture was heated at 40° C. for 4 hours then cooled, filtered and washed with water (20 mL) and then diethyl ether (20 mL) to obtain a white solid in 85% yield. Nuclear Magnetic Resonance (NMR) analysis was performed on the resultant product, with the following results, 1H NMR (400 MHz, DMSO-d₆) δ (ppm): 10.33 (s, 1H), 7.89 (d, J=8 Hz, 2H), 7.37 (d, J=8 Hz, 2H), 6.65 (s, 2H), 4.92 (s, 2H), 4.24 (t, J=4 Hz, 2H), 3.56 (t, J=4 Hz, 2H), 3.25 (s, 3H). Retention time (Rt) on HPLC=14.3 min. ESI-MS (positive ion mode): calculated for C₁₆H₁₇N₅O₅ m/z [M+1] 360.34. found 360.24.

Synthesis of 2-(4-((6-amino-2-(2-methoxyethoxy)-8-oxo-7H-purin-9(8H)-yl)methyl)benzamido)ethyl 2,3-bis(oleoyloxy)propyl phosphate (see FIG. 1, compound 6). To a solution of 0.022 g (0.06 mmol) of compound 5 in 1 mL of anhydrous N,N-dimethylmethanamide (DMF) was added 0-(7-azabenzotriazol-1-yl)-N,N,N′,N′-tetramethyluronium hexafluorophosphate (HATU) (0.026 g, 0.067 mmol) and anhydrous triethylamine (TEA) (17.0 μL, 0.12 mmol). A solution of 1,2-Dioleoyl-sn-glycero-3-phosphoethanolamine (0.05 g, 0.067 mmol) in anhydrous 1:1 dichloromethane (DCM):DMF (1 mL) was prepared and slowly added to the reaction mixture (FIG. 1 reaction step (e)). The reaction mixture was stirred at room temperature until completion and then evaporated in vacuo. The product was purified by flash chromatography using 15% methanol (MeOH) in DCM to give 0.038 g of white solid in 58% yield. NMR analysis was performed on the resultant product, with the following results, ¹H NMR (400 MHz, DMSO-d₆) δ (ppm): 9.7 (s, 1H), 7.87 (d, J=8.3 Hz, 2H), 7.32 (d, J=8.3 Hz, 2H), 6.61 (s, 2H), 5.30 (m, 4H), 5.05 (m, 1H), 4.88 (s, 2H), 4.26 (m, 4H), 4.06 (m, 1H), 3.77 (m, 4H), 3.57 (m, 2H), 3.35 (m, 2H), 3.26 (s, 3H), 2.23 (m, 4H), 1.95 (m, 8H), 1.46 (m, 4H), 1.22 (m, 40H), 0.83 (m, 6H). ESI-MS (negative ion mode): calculated for C₅₇H₉₂N₆O₁₂P m/z [M−1] 1083.35. found 1083.75.

Synthesis of 4-((6-amino-8-hydroxy-2-(2-methoxyethoxy)-9H-purin-9-yl)methyl)-N-(32-azido-3,6,9,12,15,18,21,24,27,30-decaoxadotriacontyl)benzamide (see FIG. 1, compound 7). To a solution of compound 5 (0.100 g, 0.278 mmol) in anhydrous DMF (5 mL) was added HATU (0.117 g, 0.306 mmol) and anhydrous TEA (77.014 μL, 0.556 mmol) (see FIG. 1, reaction step (f). A solution of 0-(2-aminoethyl)-O′-(2-azidoethyl)nonaethylene glycol (0.150 g, 0.306 mmol) in anhydrous DMF (1 mL) was prepared and slowly added to the reaction mixture. The reaction mixture was stirred at room temperature until completion and then evaporated in vacuo. The product was purified by flash chromatography using 5% MeOH in DCM to give 0.224 g of an opaque oil in 93% yield. Retention time on HPLC=12 minutes. NMR analysis was performed on the resultant product, with the following results, 1H NMR (400 MHz, DMSO-d₆) δ (ppm): 10.01 (s, 1H), 8.45 (t, J=5.6 Hz, 1H), 7.78 (d, J=8.3 Hz, 2H), 7.35 (d, J=8.3 Hz, 2H), 6.49 (s, 2H), 4.90 (s, 2H), 4.25 (t, J=4 Hz, 2H), 3.57 (m, 4H), 3.5 (m, 36H), 3.4 (M, 6H), 3.26 (s, 3H). ESI-MS (positive ion mode): calculated for C₃₈H₆₁N₉O₁₄ m/z [M+1] 868.94. found 868.59.

Synthesis of 3-(1-(1-(4-((6-amino-8-hydroxy-2-(2-methoxyethoxy)-9H-purin-9-yl)methyl)phenyl)-1-oxo-5,8,11,14,17,20,23,26,29,32-decaoxa-2-azatetratriacontan-34-yl)-1H-1,2,3-triazol-4-yl)propanoic acid (see FIG. 1, compound 8). Compound 7 (0.218 g, 0.251 mmol) and 4-pentynoic acid (0.074 g, 0.753 mmol) were dissolved in 1:1 t-butanol:H₂O (3 mL) (see FIG. 1, reaction step (g)). Sodium ascorbate (0.02 g, 100 mmol) and Cu(OAc)₂ (0.009 g, 50 mmol) in 1:1 t-butanol:H₂O (1 mL) was slowly added to the reaction mixture and stirred at room temperature until compound 7 was fully reacted by TLC. The product was extracted with DCM (10 mL) and H₂O (10 mL) and the organic layer was dried over MgSO₄ to give 0.230 g of an opaque oil in 95% yield. Retention time on HPLC=11.5 minutes. NMR analysis was performed on the resultant product, with the following results, ¹H NMR (400 MHz, DMSO-d₆) 8 (ppm): 13.48 (s, 1H), 7.76 (d, J=8.29 Hz, 2H), 7.75 (s, 1H), 7.23 (d, J=8.29, 2H), 4.88 (s, 2H), 4.41 (t, J=5.12 Hz, 2H), 4.23 (t, J=4 Hz, 2H), 3.74 (t, J=5.12 Hz, 2H), 3.57 (t, J=4 Hz, 2H), 3.51 (m, 8H), 3.42 (m, 36H), 3.26 (s, 3H), 2.79 (t, J=7.56 Hz, 2H), 2.24 (t, J=7.56 Hz, 2H). ESI-MS (positive ion mode): calculated for C₄₃H₆₇N₉O₁₆ m/z [M+l] 966.04. found 966.67.

### Example II

Various purines, pyridines, and imidazoquinolines, with molecular weights of 200-400 kD, have been shown to activate TLR7 and compounds that were specific TLR7 ligands were 100-1000 fold more powerful than imiquimod on a molar basis (Lee et al., infra). Because these TLR agonists are structurally very similar to normal component of nucleotides, they are very unlikely to induce a haptenic immune reaction after repeated administration.

### Experimental Methods

### In Vitro Methods

In vitro measurements of cytokine induction were performed using the mouse leukemic monocyte macrophage cell line, RAW264.7. Raw264.7 mice were obtained from the American Type Culture Collection (ATCC, Rockville, Md.) and cultured in DMEM complete media [Dulbecco's Modified Eagle Medium (Irvine Scientific, Irvine, Calif.) supplemented with 10% heat-inactivated fetal calf serum, 2 mM L-glutamine, and 100 U/mL penicillin/100 μg/mL streptomycin]. BMDM were prepared from C57BL/6 and TLR7 deficient mice as described in Wu, C. C et al., “Immunotherapeutic activity of a conjugate of a Toll-like receptor 7 ligand”, Proc. Natl. Acad. Sci. USA, 104:3990 (2007).

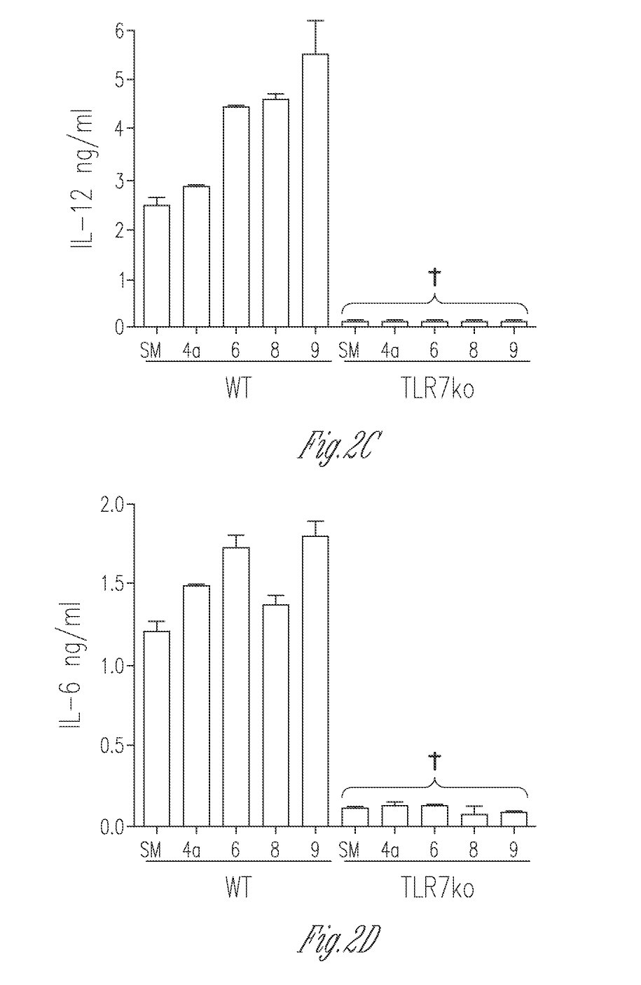

In general, RAW264.7 cells or BMDM were incubated with various concentrations of conjugates for 18 hours at 37° C., 5% CO₂ and culture supernatants were collected. The levels of cytokines (IL-6, IL-12 or TNF-α in the supernatants were determined by ELISA (BD Biosciences Pharmingen, La Jolla, Calif.) (Cho, H. J et al., “Immunostimulatory DNA-based vaccines induce cytotoxic lymphocyte activity by a T-helper cell-independent mechanism” [see comments], Nat. Biotechnol., 18:509 (2000)), and the results presented in FIGS. 2A-D. Data are mean±SEM of triplicates and are representative of three independent experiments. Minimum detection levels of these cytokines were 15 pg/mL.

TNFα levels were measured (see FIG. 2A) by incubating approximately 1×10⁶/mL RAW 264.7 cells with the various conjugates or controls, as described above. IL-6 and IL-12 levels were measured (see FIGS. 2B-D) by incubating 0.5×10⁶/mL BMDM with the various conjugates or controls, as described above. Conjugates were prepared as stock solutions (10 μm for SM, and compounds (6), (8), and (9), 0.1 μm for compound (4a)), and serial dilutions (1:5) prepared therefrom.

BMDM were also used to evaluate the level of endotoxin contamination of TLR7 conjugates synthesized using synthesis schemes described herein. 0.5×10⁶/mL BMDM derived from C3H/HeJ (LPS unresponsive mutant) or C3H/HeOuJ (wild type) were incubated with TLR7 conjugates (10 μM SM, 0.1 μM compound 4a, 10 μM compound 6, 10 μM compound 8 or 10 μM compound 9) for 18 hours. IL-6 or IL-12 levels in culture supernatants were measured by ELISA, and the results presented in FIG. 2B. Each of the TLR7 conjugates induced similar levels of IL-6 both in TLR4 mutant and wild type mice, indicating LPS contamination of these conjugates is minimal.

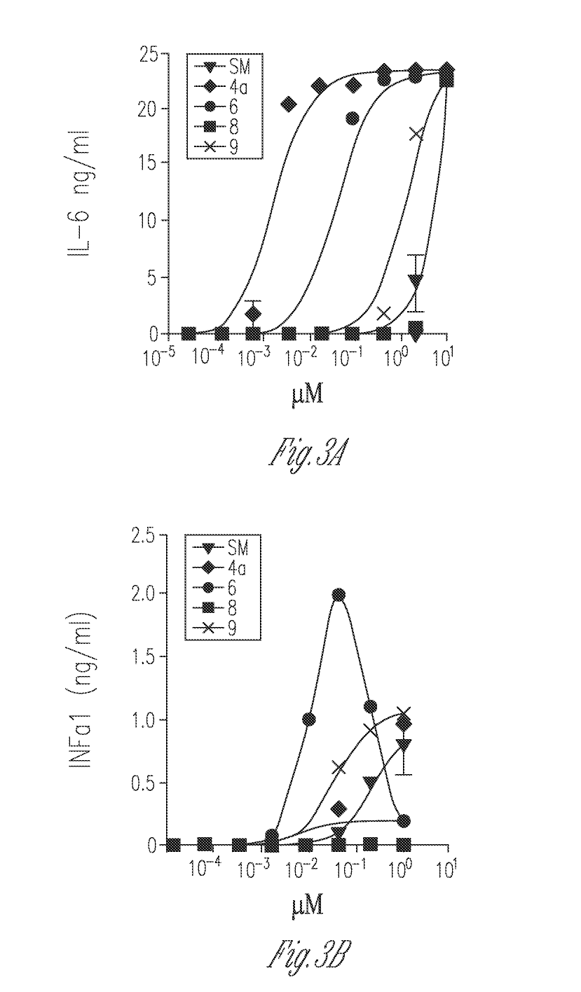

Human blood peripheral mononuclear cells (PBMC) were isolated from human buffy coats, purchased from The San Diego Blood Bank (San Diego, Calif.), as described in Hayashi, T et al., “Enhancement of innate immunity against Mycobacterium avium infection by immunostimulatory DNA is mediated by indoleamine 2,3-dioxygenase”, Infect. Immun., 69:6156, (2001). PBMC (1×10⁶/mL) were incubated with various concentrations of TLR7 conjugates for 18 hours at 37° C., 5% CO₂ and culture supernatants were collected. The levels of cytokines (IL-6, TNF-μ, or IFNα1) in the supernatants were determined by Luminex bead assays (Invitrogen, Carlsbad, Calif.), and the results presented in FIG. 3A-B. Data are mean±SEM of triplicates and are representative of three independent experiments. The minimum detection levels of IL-6, TNF-α, and IFNα1 were 6 pg/mL, 10 pg/mL and 15 pg/mL, respectively.

### In Vivo Methods

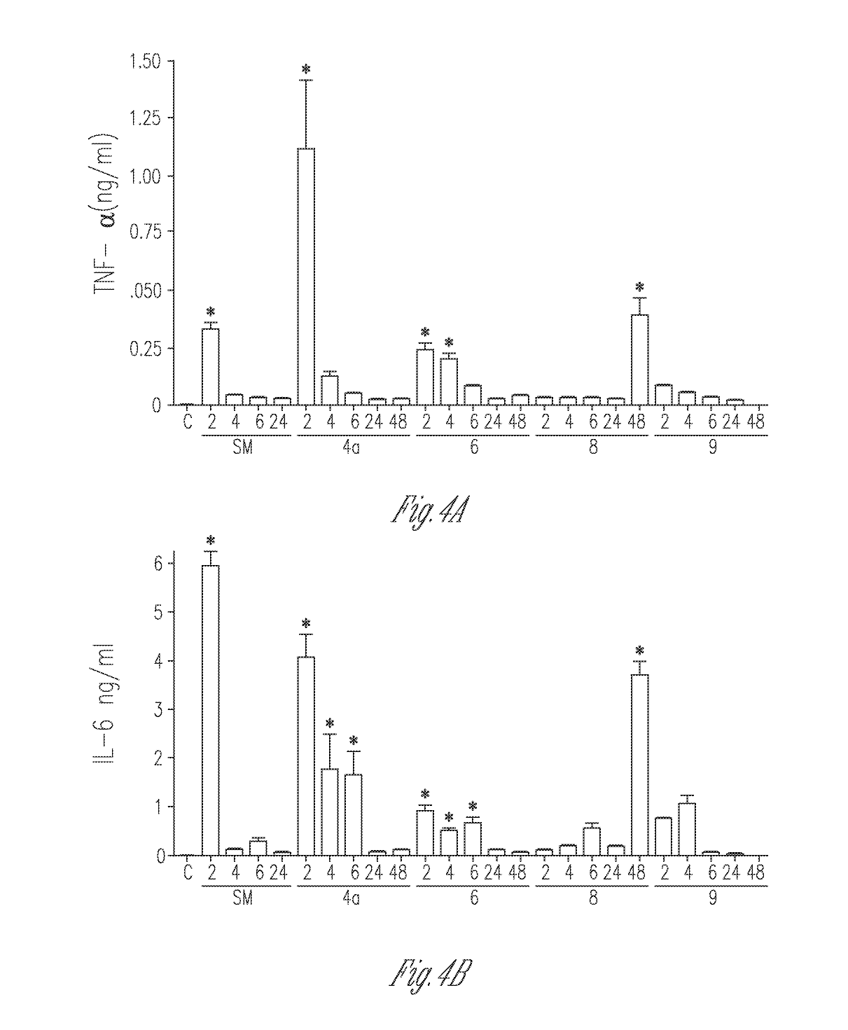

The pharmacokinetics of pro inflammatory cytokine induction by TLR7 conjugates was examined using 6- to 8-week old C57BL/6 mice. The mice were intravenously injected with TLR7 agonists and their conjugates (40 nmol compound (4a) or 200 nmol SM and compounds (6), (8), or (9) per mouse). Blood samples were collected 2, 4, 6, 24 or 48 hours after injections. Sera were separated and kept at −20° C. until use. The levels of cytokines (e.g. IL-6 and TNF-α in the sera were measured by Luminex bead microassay, and the results presented in FIGS. 4A-B. Data are mean±SEM of five mice and are representative of two independent experiments. The minimum detection levels of IL-6 and TNF-α are 5 pg/mL and 10 pg/mL, respectively.

Immunological reaction initiation (e.g., adjuvanticity) by TLR7 conjugates was also examined. Groups (n=5) of C57BL/6 mice were subcutaneously immunized with 20 μg ovalbumin (OVA) mixed with approximately 10 nmol of various TLR7 conjugates, on days 0 and 7, where 10 nmol is a dosage target for the TLR7 portion of the conjugate, and the actual amount will be dependent on the actual chemical formula of each conjugate. A TLR9-activating immunostimulatory oligonucleotide sequence (ISS-ODN; 1018) was used as a positive control for a Th1 inducing adjuvant (Roman, M et al., Immunostimulatory DNA sequences function as T helper-1-promoting adjuvants, Nat. Med., 3:849 (1997)). Sera were collected on days 0, 7, 14, 21, 28, 42 and 56. Mice immunized with saline or OVA mixed with vehicle served as controls. Mice were sacrificed on day 56 and the spleens were harvested for preparation of spleenocytes and histological slides. Approximately 200 microliters of a 2.5×10⁶/mL spleen cell stock were aliquoted into round-bottom tissue culture microtiter plates in triplicate in a total volume of 200 μl RPMI 1640 complete medium [RPMI1640 (Irvine Scientific, Irvine, Calif.) supplemented with 10% heat-inactivated FCS, 2 mM L-glutamine, and 100 U/mL penicillin/100 μg/mL streptomycin] and restimulated with either 100 μg/mL OVA or medium alone. In some experiments the site of injection was examined 24 hours after immunization for signs of inflammation or local reaction. Mice were observed for activity as measures of a potential “sickness” response to immunization and then weighed weekly. In addition to spleen harvesting, lungs, livers, hearts, and kidneys also were collected on day 56, fixed in 10% buffered Formalin (Fisher Scientific, Pittsburgh, Pa.) and embedded in paraffin. Sections 5 μm thick were stained with hematoxylin and eosin (H&E) and evaluated under the microscope.

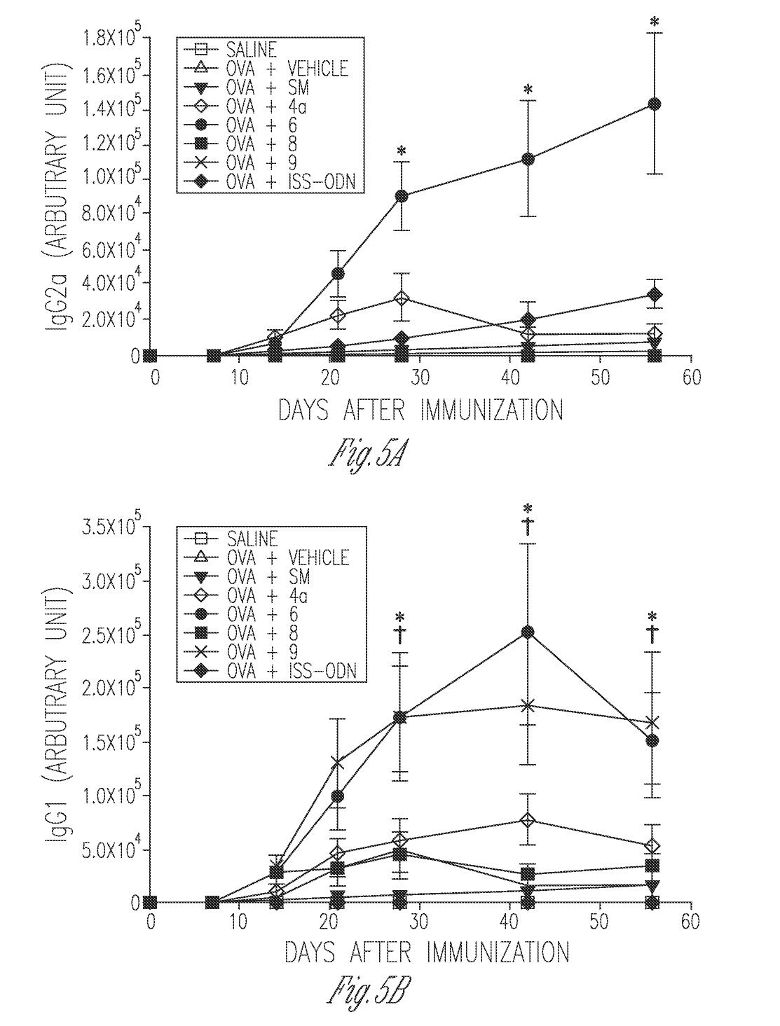

Anti-OVA antibodies of the IgG subclasses (and in some embodiments specifically IgG1 and IgG2) were measured by ELISA, as described in Cho, H. J et al., “Immunostimulatory DNA-based vaccines induce cytotoxic lymphocyte activity by a T-helper cell-independent mechanism” [see comments], Nat. Biotechnol., 18:509 (2000), and the results presented in FIGS. 5A and 5B. Each ELISA plate contained a titration of a previously quantitated serum to generate a standard curve. The titer of this standard was calculated as the highest dilution of serum that gave an absorbance reading that was double the background. The various sera samples were tested at a 1:100 dilution. The results are expressed in units per mL, calculated based on the units/mL of the standard serum, and represent the mean±SEM of five animals in each group. * and T denote P<0.05 and P<0.01 by One-way ANOVA compared to the mice immunized with OVA mixed with vehicle, respectively.

Spleenocytes were prepared from the harvested spleens. Spleenocyte cultures (restimulated either 100 μg/mL OVA or medium alone) were then incubated at 37° C., 5% CO₂ and supernatants harvested after 72 hours. The levels of IFNα in the culture supernatants were measured by ELISA (BD Bioscience PharMingen) as per the manufacturer's instructions (Kobayashi, H et al., Prepriming: a novel approach to DNA-based vaccination and immunomodulation”, Springer Semin. Immunopathol., 22:85 (2000)), and the results illustrated in FIG. 5C. Average total spleen cell number in each group were calculated and compared to the PBS-immunized groups to monitor the spleen cell proliferation. Data are mean±SEM of five mice and are representative of three independent experiments.

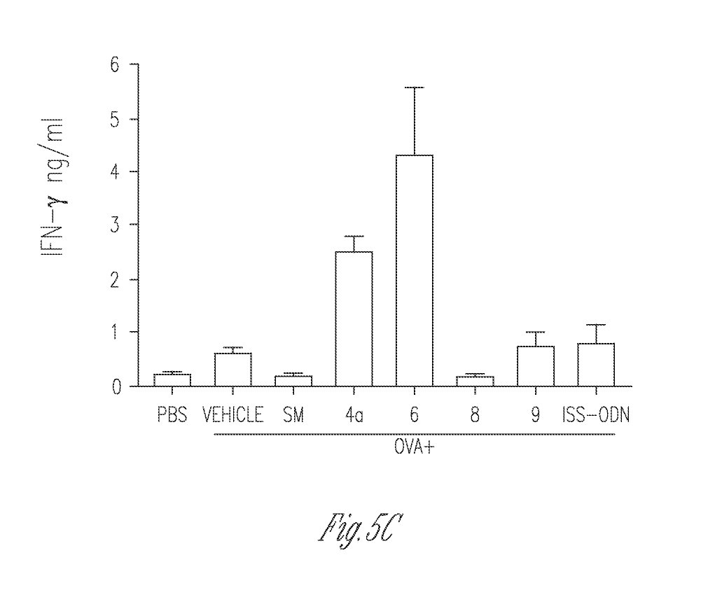

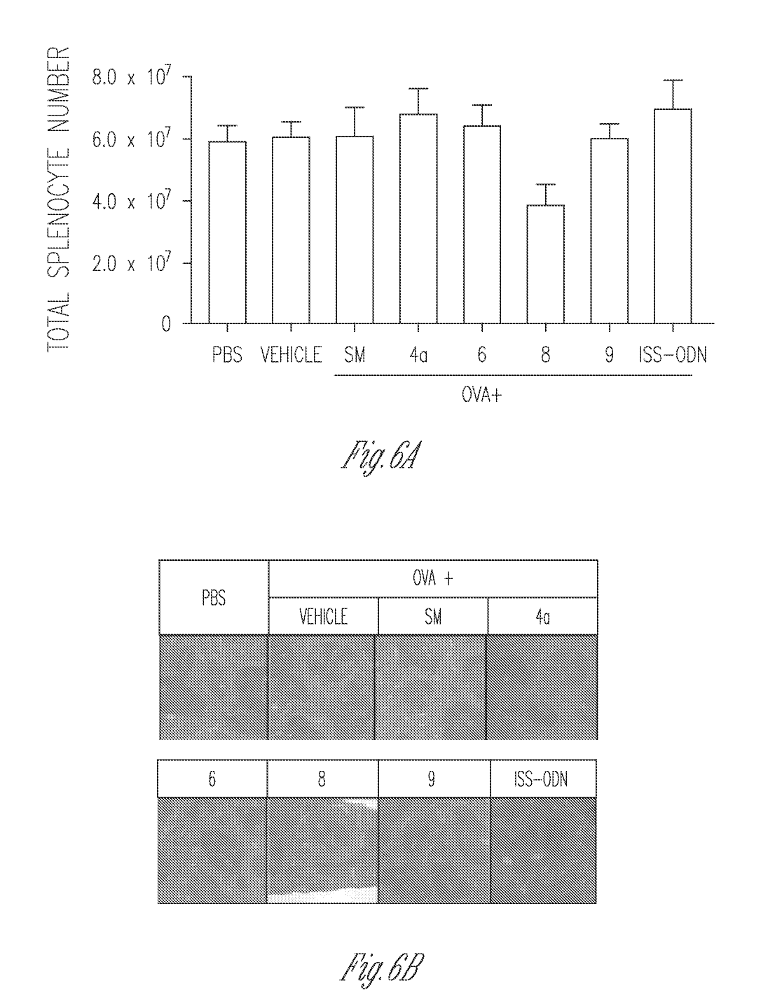

Evaluation of possible adverse effects of TLR7 conjugates was performed by a three-fold analysis (counting of total spleenocytes, histological examination, and visual observation of both the area of injection and general overall health and behavior of treated mice). C57BL/6 mice were immunized with 20 μg OVA mixed with TLR7 conjugate, vehicle, or a control agonist (oligonucleotide sequence ISS-ODN). On day 56, mice were sacrificed and number of total spleenocytes was counted, and the results presented in FIG. 6A. The spleens were collected and submitted to the histological examination, as shown in FIG. 6B (magnification factor=100×). The skin of injection sites is inspected 24 hours after injection, as shown in FIG. 6C. There is no significant difference in the number of splenocytes counted between mice immunized with OVA plus TLR7 conjugates and the mice immunized with OVA alone (see FIG. 6A). Histological examination of the spleens from mice immunized with OVA mixed with TLR7 conjugates did not show any disruption of the white pulps or increased cellularity in red pulp (see FIG. 6B). The skin of injection sites did not have visible redness or glaucomatous reaction (see FIG. 6C).

In some experiments statistical evaluation was performed to determine the statistical significance of the observed results. A statistical software package (Prism 4.0, GraphPad, San Diego Calif.) was used for statistical analyses including regression analysis. Data were plotted and fitted by non-linear regression assuming a Gaussian distribution with uniform standard deviations between groups. In the adjuvanticity experiments, the statistical difference between the groups were analyzed by two way ANOVA with Bonferroni post-tests to compare control mice with those that were immunized with OVA. A value of P <0.05 was considered statistically significant.

### Results and Discussion

### Chemical Synthesis

The synthesis of compound (4) from compound (1) yielded a consistent conjugation ratio of 5:1 UC1V150 to MSA protein (Wu, C. C et al., “Immunotherapeutic activity of a conjugate of a Toll-like receptor 7 ligand”, Proc. Natl. Acad. Sci. USA, 1.04:3990 (2007)). Basic hydrolysis (FIG. 1, reaction step (d)) of the 9-benzylnitrile of compound (1) provided a versatile benzoic acid functional group (compound (5)) and allows for the assembly of conjugates (6), (8), and (9). The benzoic acid was coupled with DOPE by activation with HATU in the presence of TEA in anhydrous DMF (FIG. 1, reaction step (e)) to give compound 6 in 58% yield.

Due to the difficulty in dissolution of compound (6) in suitable solvents for testing, a PEG spacer was coupled to provide improved solubility. A readily available amine/azide bifunctional PEG was coupled to the benzoic acid by activation with HATU in the presence of TEA in anhydrous DMF (see FIG. 1, reaction step (f), which results in compound (7)). The formation of a 1,2,3-triazole through a copper(I)-catalyzed azide-alkyne cycloaddition with 4-pentynoic acid (FIG. 1, reaction step (g)) gave compound (8) in 95% yield. Finally, compound (9) was prepared by HATU activated amide formation with DOPE (FIG. 1, reaction step (h)) and compound (8).

### In Vitro Measurement of Cytokine Induction by Lipid-Conjugated TLR7 Agonists

TLR7 agonist compound (4a), when covalently coupled with mouse serum albumin, exhibited a potency of 10 or higher in cytokine induction in vitro and in vivo compared to unconjugated drug (SM) (Wu, C. C et al., “Immunotherapeutic activity of a conjugate of a Toll-like receptor 7 ligand”, Proc. Natl. Acad. Sci. USA, 104:3990 (2007)). Using a similar assay, the in vitro potency of the lipid-TLR agonist conjugates (FIG. 1, compound 6), PEG-TLR7 agonist conjugates (FIG. 1, compound 8), and PEG-lipid (FIG. 1, compound 9) conjugates were compared using a murine macrophage cell line, RAW264, and primary bone marrow derived macrophages (BMDM). The respective cells were stimulated for 18 hours with serially diluted TLR7 conjugates and the levels of cytokines released in the media were measured by ELISA and compared to the unconjugated TLR7 agonist (SM) (see FIG. 2A, panels A-D).

Compound (4a) (e.g., a TLR7-MSA conjugate) was previously shown to be 100-fold more potent as a cytokine inducer, when compared to the unconjugated agonist, whereas the Lipid-TLR7 conjugate was 10-fold more potent, when normalized to the molar level of the unconjugated agonist. Although the PEG-TLR7 conjugates (compound 8) showed less potency compared to the unconjugated TLR7 (SM), conjugation of lipid to PEG-TLR7 conjugates (lipidPEG-TLR7) (compound 9) restored their potency to the similar level of the unconjugated TLR7 (SM). Substantially similar concentrations of MSA, lipid or PEG without TLR7 conjugation, at the highest levels in the conjugated form, were used as a negative control and induced minimal or no cytokine levels in RAW264.7 cells and BMDM, respectively (data not shown).

In order to evaluate if the conjugated forms of TLR7 agonists were solely inducing macrophage stimulation, as opposed to non-TLR7 macrophage stimulation, BMDM derived from wild type and TLR7 deficient mice (TLR7-KO or knock out mice) were treated with compounds (4a), (6), (8), (9) and SM. Compounds (4a), (6), (8), (9) and SM, induced little or no IL-12 and IL-6 whereas these conjugates were active in wild type BMDM, indicating the agonist activity was due to the TLR7 activity of these conjugates (see FIGS. 2C-D). Endotoxin evaluation (FIG. 2B, and described above) further supported the conclusion that the agonist activity was due to the TLR7 activity of these conjugates (e.g., no significant statistical difference in the levels of IL-6 produced).

To further investigate the immunological activities in human cells, human PBMC from three donors were treated with TLR7 conjugates and the levels of IL-6 and IFNα1 were determined by Luminex assay (FIGS. 3A-B). Human serum albumin (HSA) conjugated to TLR7 (4b) was used instead of MSA-conjugates (4a) in this experiment. The order of TLR7 conjugate potency was similar to the order observed in murine macrophages ((4b)>(6)>(9)>/=SM>(8)) (FIG. 3A). A consistent trend in compound potency was observed in PBMC from all donors. Unlike Compound (4a), compound (4b) (e.g., TLR7-HSA conjugate) induced minimum levels of IFNa1 in human PBMC (observed in three donors) (FIG. 3B).

### In Vivo Kinetics of Induction of Pro-Inflammatory Cytokines by TLR Conjugates

To compare the in vivo immunological properties of TLR7 conjugates, C57BL/5 mice received TLR7 agonist conjugates intravenously and the kinetics of pro-inflammatory cytokines in sera were studied (FIGS. 4A and 4B). Based on a previous study (Wu, C. C et al., Proc. Natl. Acad. Sci. USA, 104:3990 (2007)), compound (4a) was used at a lower concentration (40 nmol per animal) than compounds SM, (6), (8) and (9) (200 nmol per animal). The maximum induction of TNFα and IL-6 were observed at 2 hours post injection for all TLR7 conjugates (FIGS. 4A-B, respectively). The levels of the cytokines induced by unconjugated TLR7 (SM) declined rapidly after 2 hours. Cytokine induction by compounds (4a), (6), and (9), were sustained for up to 6 hours.

Compound (8) induced only a low level of IL-6 (see FIG. 4B), and had no significant induction of TNFα, at any point post injection (see FIG. 4B). Sera from control mice that received saline, MSA, or DOPE revealed little or no detectable cytokine levels (data not shown).

### Lipid-TLR7 Conjugates Promote Rapid and Long Lasting Humeral Responses

The efficiency of adjuvanticity was assessed by measurement of the levels and isotypes of the antigen-specific IgG that the vaccine induces, in particular IgG1 and IgG2 (Mosmann, T. R., and Coffman, R. L., ‘TH1 and TH2 cells: different patterns of lymphokine secretion lead to different functional properties’, Annual Review Immunology, 7:145 (1989)). The groups of C57BL/6 mice (n=5 animals per group) were subcutaneously immunized with OVA (ovalbumin) mixed with TLR7 conjugates. ISS-ODN was used as a potent Th1 adjuvant positive control. Mice immunized with saline or OVA plus vehicle (0.1% DMSO) were used as negative controls. OVA-specific IgG1 and IgG2a serum induction kinetics were monitored by ELISA, on days 0, 7, 14, 21, 28, 42, and 56 (FIGS. 5A-B). Induction of antibodies of the IG subclass was observed as early as 14 days in mice immunized with OVA mixed with compound (4a) or compound (6) (see FIG. 5A). The anti-OVA IgG2a levels continuously increased in mice immunized with OVA/compound (6) mixtures, whereas the levels in mice immunized with OVA/compound (4a) mixture subsequently declined, as illustrated in FIG. 5A. These data are consistent with the enhanced OVA-specific IFNα secretion by spleen cells of mice immunized with OVA combined with compounds (4a) or (6) (see FIG. 5C).

### In Vivo Evaluation of Adverse Effects

TLR7 agonists (SM) can induce anorexic effects and hypothermia in mice (Hayashi, T et al., “Mast cell dependent anorexia and hypothermia induced by mucosal activation of Toll like Receptor 7”, Am. J. Physiol. Regul. Integr. Comp. Physiol., 295:R123 (2008)), causing weight loss in mice. Therefore, as part of the experimental protocol, body weight and skin reaction (at site of injection) of the mice immunized, with lipid-TLR7 agonist conjugates, was monitored. The minimum dose of unconjugated TLR7 agonist (SM) that induced the anorectic reaction in mice was 50 nmoles per mice in mucosal administration (Hayashi, T et al., Am. J. Physiol. Regul. Integr. Comp. Physiol., 295:R123 (2008)). The dose for the adjuvant experiments (10 nmoles per mouse) was selected to avoid the sickness reaction caused by TLR7 agonists. No significant differences were observed between the average body weights of mice immunized with OVA mixed with compound (6) and the mice injected saline (data not shown).

Chronic administration of TLR7 can also induce myeloid cell proliferation (Baenziger, S et al., “Triggering TLR7 in mice induces immune activation and lymphoid system disruption, resembling HIV-mediated pathology”, Blood, 113:377 (2009). Total number of spleen cells was calculated as an indicator of the splenic myeloid cell proliferation (see FIG. 6A). There was no significant difference in the total number of spleenocytes between the mice immunized with OVA, TLR7 agonist conjugates and saline control (see FIG. 6B). Histological examination of spleens from mice immunized with OVA mixed with TLR7 agonist showed no structural disruption of the white pulp (germinal center) and no increased cellularity in red pulp (see FIG. 6B). Additionally, no significant difference was observed in the histological examination of the liver, lung, heart and kidney samples collected from each group (data not shown). There also was no macroscopically visible redness or glaucomatous reaction at or near the site of injection with lipid-TLR7 conjugates (see FIG. 6C).

### CONCLUSIONS

Unconjugated TLR7 (SM) is insoluble in aqueous solution. Water-solubility can play a role in controlling drug availability by increasing drug diffusion or promoting uptake to the cells. PEGylation can improve drug solubility and decrease immunogenicity (Veronese, F. M., and Mero, A, “The impact of PEGylation on biological therapies”, BioDrugs, 22:315 (2008)). PEGylation can also increase drug stability, the retention time of the conjugates in blood and can reduce proteolysis and renal excretion (Veronese, F. M., and Mero, A, BioDrugs, 22:315 (2008)). When TLR7 is conjugated to PEG (e.g., compound (8)), the solubility improves dramatically (data not shown). However, potency of cytokine induction is attenuated in comparison to the unmodified TLR7 agonist, in vitro (FIG. 2A, panel A and B) and in vivo (FIGS. 4A and 4B). Activity in both in vitro and in vivo can be restored by further conjugation to DOPE (compound (9)). Compound (9) can induce a Th2 immune response (indicated by IgG1 levels), while exhibiting minimal Th1 response (indicated by IgG2a levels).

TLR7 agonist conjugates compounds (4a) (MSA conjugate) and (6) (lipid conjugate) promoted rapid elevation of IgG2a titer (FIG. 5A). Levels of IgG2a in mice immunized with MSA-TLR7 conjugates (compound (4a)) declined three weeks after the last immunization, while the mice immunized with OVA mixed with lipidTLR7 conjugates (compound (6)) showed sustained and further accelerated levels of antigen-specific IgG2a (FIG. 5A). Although compound (4a) failed to maintain the levels of IgG2a, the secretion of OVA-specific IFN-gamma by spleen cells in mice immunized with OVA mixed with compound (4a) maintained relatively high levels (FIG. 5C). The same TLR7 agonists conjugated to different moieties that give distinct immune profiles, can be useful in the design of adjuvants to treat distinct disease categories, such as infection and autoimmune disease, for example.

Various conjugates of a TLR7 agonist were synthesized and found to have distinct immunological profiles both in vivo and in vitro. Diversity in physical properties of reported TLR7 agonist conjugates may allow for a broader range of applications in treatment of different diseases. Water-soluble conjugates can provide a route for systemic administration. Lipid containing conjugates may be suitable for local administration requiring persistent stimulation of the adjacent immune cells (e.g., application of adjuvant for infectious diseases). A lipid moiety may facilitate drug penetration through the epithelium of the bladder or the skin and so may be beneficial for treatment of bladder or skin disorders. Conjugation of TLR7 agonist to lipid or PEG moieties may be a promising strategy to expand clinical treatment of infection, cancer or autoimmune disease.

Activation of Toll-like receptors (TLRs) on cells of the innate immune system initiates, amplifies, and directs the antigen-specific acquired immune response. Ligands that stimulate TLRs, therefore, represent potential immune adjuvants. Each conjugate having a potent TLR7 agonist conjugated with polyethylene glycol (PEG), lipid, or lipid-PEG via a versatile benzoic acid functional group may display distinctive immunological profiles in vitro and in vivo. For example, in mouse macrophages and human peripheral blood mononuclear cells, the lipid-TLR7 conjugates were at least 100 fold more potent than the free TLR7 ligands. When the conjugates were administered systemically in vivo, the lipid and lipid-PEG TLR7 conjugates provided sustained levels of immunostimulatory cytokines in serum, compared to the unmodified TLR7 activator. These data show that the immunostimulatory activity of a TLR7 ligand can be amplified and focused by conjugation, thus potentially broadening the potential therapeutic application of these agents.

### Example III

### Induction of Bladder Inflammation by 1V270

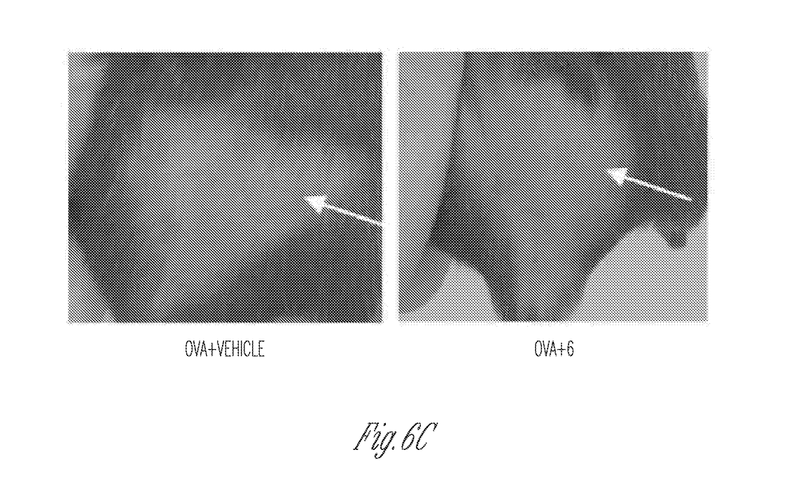

C57BL/6 mice or caspase 1 deficient were intravesically treated with 150 nmoles 1V270 or vehicle alone three times at two-day intervals. One set of mice was sacrificed twenty-four hours after the first treatment (FIG. 7). Another set of mice was sacrificed twenty-four hours after the third treatment. After the initial treatment, the infiltration of mononuclear cells were observed and increased after the third treatment. This inflammation was not detected in the caspase 1 deficient mice.

### Systemic Cytokine Induction by Dermal Application of 1V270

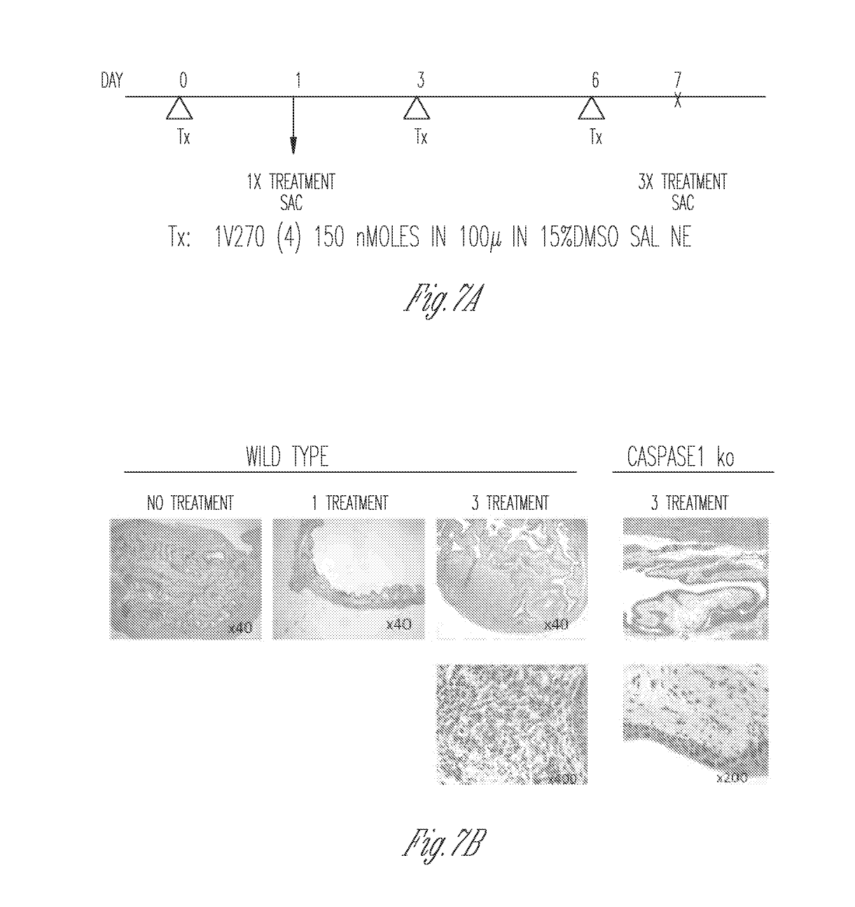

5% 1V270 in Aquaphor was prepared and the pharmacodynamics was compared to the 5% Aldara cream obtained from the pharmacy at UCSD Moores Cancer Center. One day before the study, 6 to 8 week old female C57BL/6 mice were shaved on flank. The ointment/cream was applied on a one square inch area. Sera were collected at 2, 4, 6, 24 and 48 hours after the application. 5% 1V270 systemically induced comparable levels of IL-6 and TNFα to those of Aldara cream (5%) (FIG. 8).

### Pulmonary (Mucosal) Application of 1V270 to Prevent Anthrax Infection

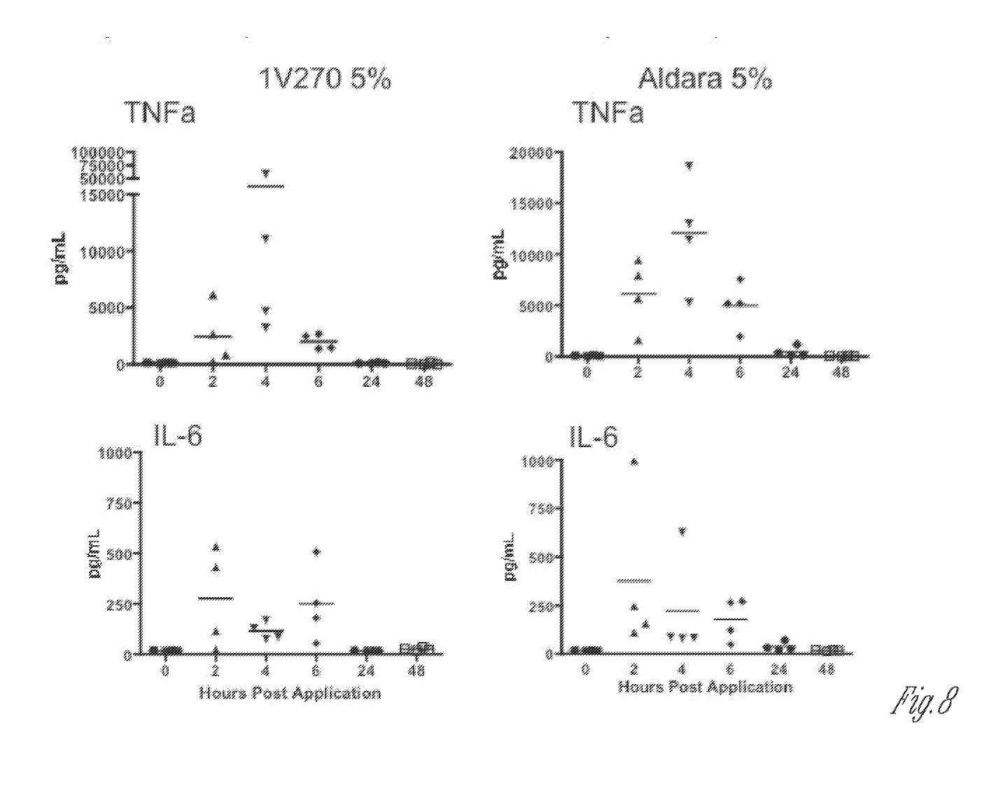

1V270 induces local inflammation by pulmonary administration. To study the ability of phospholipid conjugated TLR7 to initiate the local inflammation, 6 to 8-week old female A/J mice were intranasally administered with 1V270 (0.5, 1, 2 or 4 nmol per animal) in 5% DMSO in PBS. Mice were sacrificed two hours after the administration and serum and bronchial lavage fluid (BALF) were collected. The levels of IL-6, IL-12 and TNFα were determined by Luminex beads assay. 2 to 4 nmol 1V270 induced local proinflammatory cytokines in the BALF (FIG. 9). The levels of these cytokines were higher than those in the serum.

The Inflammation Induced by 1V270 is Persistent.

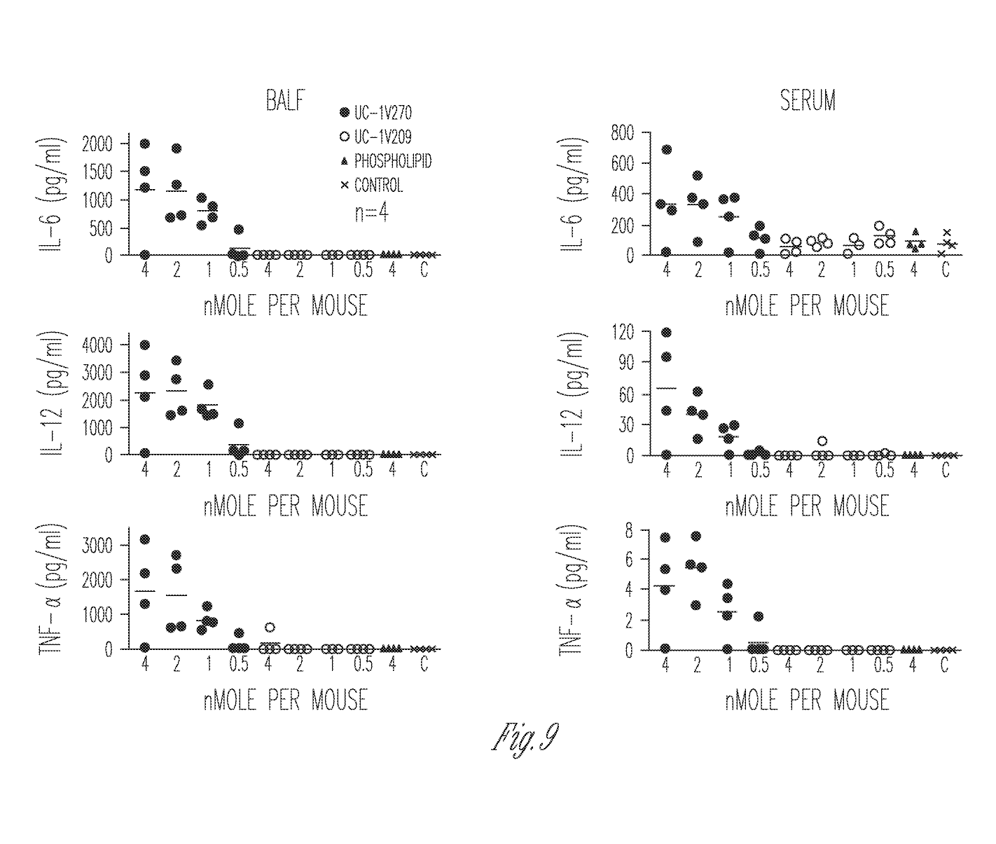

It was further investigated if the inflammation initiated by 1V270 persists longer than unconjugated TLR7 agonist. A/J mice received 10 nmol/animal 1V270 intranasally and BALF were collected 24, 48, and 72 hours after the administration. 1V270 could induce IL-12 and TNFα up to 48 hours (FIG. 10).

Vaccine Application of 1V270 to Prevent Anthrax Infection.

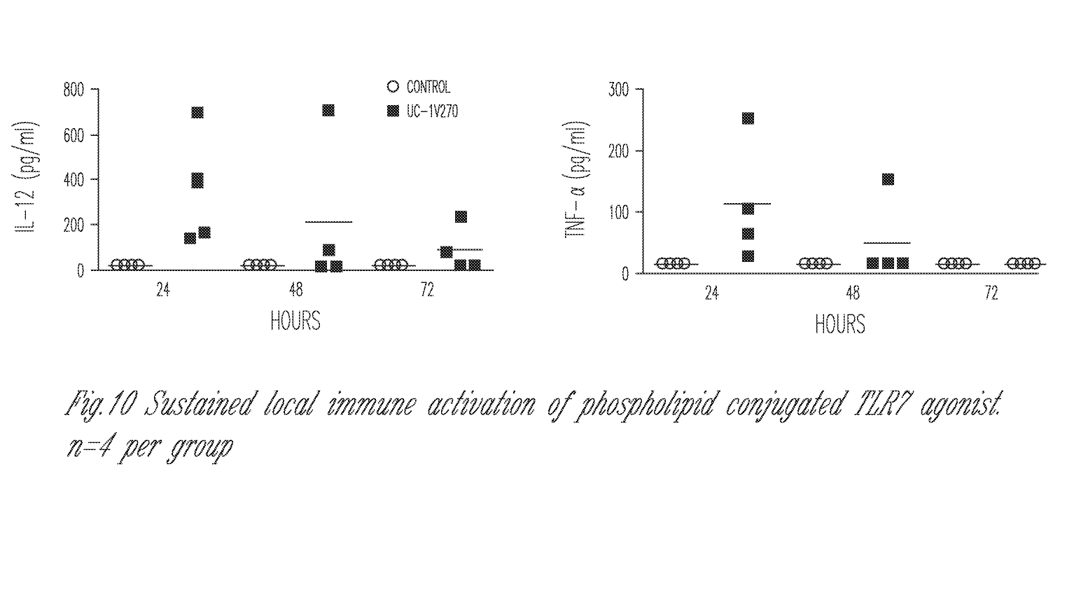

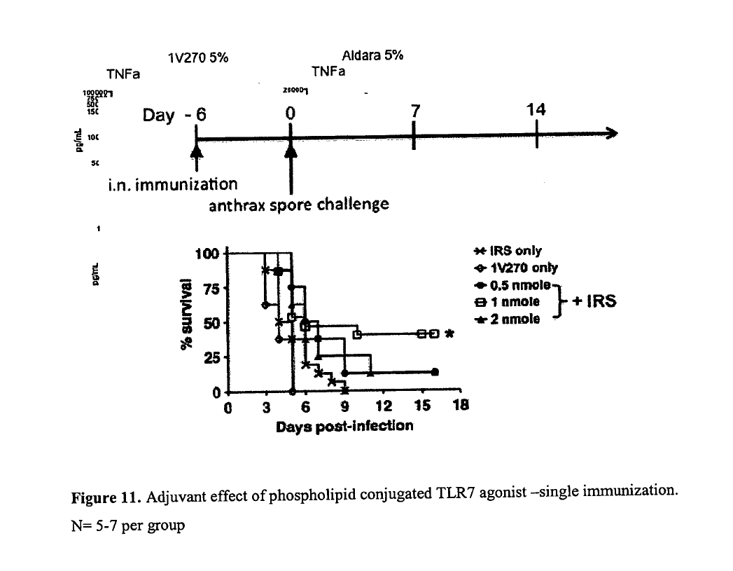

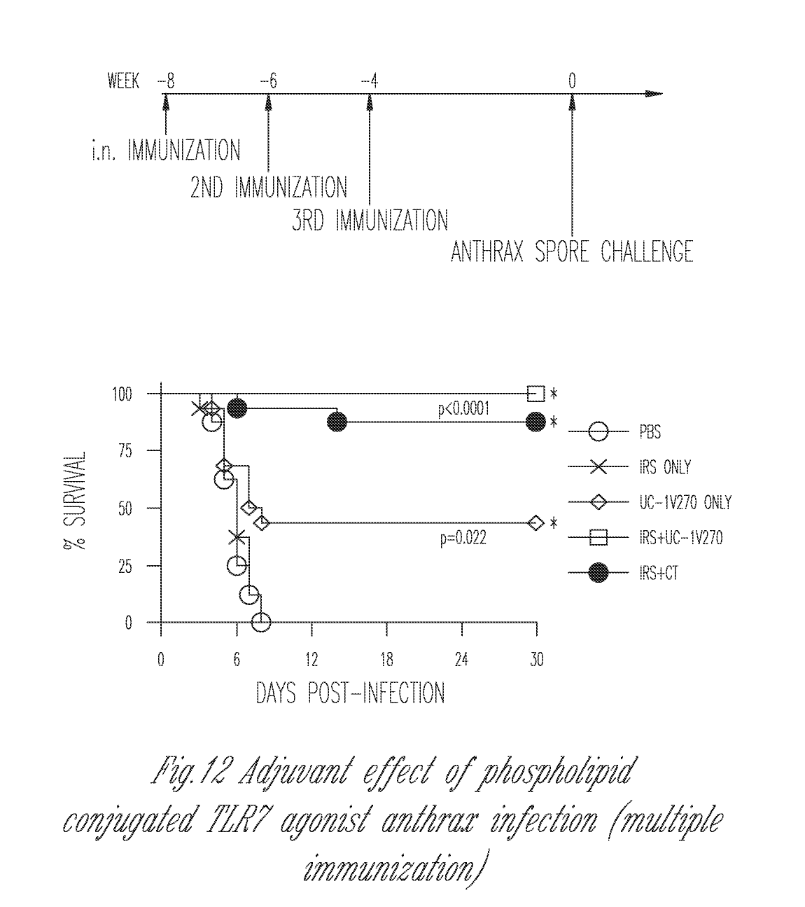

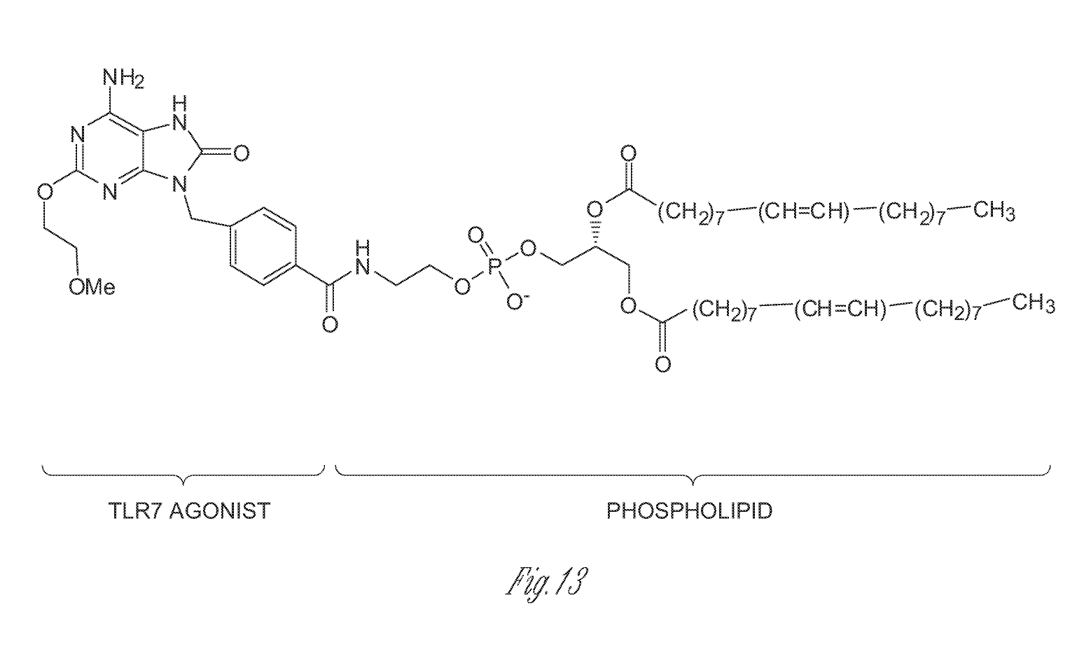

1V 270 is a potent adjuvant when administered with the antigen, ovalbumin. To evaluate the adjuvant efficacy of 1V270 against Anthrax infection, mice were administered with irradiated Anthrax spores (IRS) (Wu et al., PNAS, 104:3990 (2007)) mixed with 1V270. In order to optimize the schedule of vaccination, two experiments were performed; short term (FIG. 11) and long term (FIG. 12) experiments.

In the short term experiment (FIG. 11), A/J mice were treated with IRS with 0.5, 1 or 2 nmol 1V270 and challenged with Anthrax Sterne strain spores six days after the vaccination. Control mice received IRS or 1V270 (4 nmol/animal). More than half of the mice treated with 1 nmol 1V270 mixed with IRS survived on day 15, but not 0.5 nmol or 2 nmol. Therefore, 1 nmol per animal was used in the subsequent long term experiment.

In the long term experiment (FIG. 12), the mice were treated with 1V270 mixed with IRS intranasally three times at two week intervals. Cholera toxin was used as a positive control because it is a known effective mucosal adjuvant. Control mice were treated with 1V270, IRS or vehicle (PBS). All mice were intranasally challenged with Anthrax spores four weeks after the last vaccination. 100% survival on day 30 was observed in mice vaccinated with IRS mixed with 1V270, which was slightly more effective than mice that received CT as an adjuvant.

### Example IV

### Nanoparticle Formulations of 1V270

Phosal 50 PG Formulation.

1V270 was dissolved in Phosal 50 PG (Phospholipid Gmbh, Cologne, Germany) to make a 20× concentrated solution. The Phosal 50 PG-1V270 mixture was further diluted (1:19) with nanopure water to make a 5% Phosal 50 PG:water suspension. The suspension was vortexed vigorously and sonicated in a sonicating bath for 10 minutes. The suspension was further sonicated with a probe sonicater (Branson Sonifier Cell Disrupter 185) at 30% power for a total of 30 seconds at 10 second intervals with 10 seconds rest between so as to not overheat the suspension. Finally, the suspension was passed through a 100 nm filter with syringe extruder a total of 10 times back and forth. The final nanoparticles were analyzed with a Malvern Zetasizer to check size distribution. The resulting particles may be referred to as nanoliposomes (a submicron bilayer lipid vesicle) (see Chapter 2 by Mozafari in: Liposomes, Methods in Molecular Biology, vol. 605, V. Weissing (ed.), Humana Press, the disclosure of which is incorporated by reference herein). Nanoliposomes provide more surface area and may increase solubility, bioavailability and targeting.

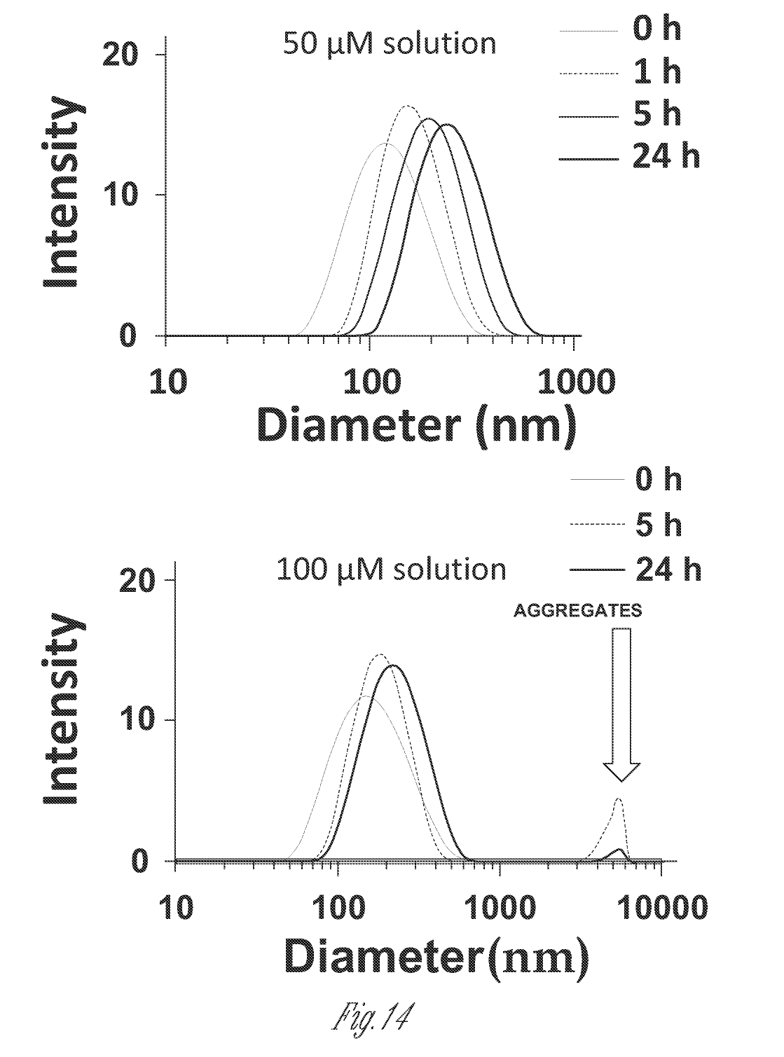

UV-1V270 particles were diluted in PBS to 50 μM (A) or 100 μM (B) and particle size measured over time. As shown in FIG. 14, the nanoparticles were generally stable over time. Some aggregates were observed at 100 μM, which is about the upper limit of solubility. The particle size of UV-1V270 in PBS was relatively constant with an average of about 110 nm regardless of concentration.

### Example V

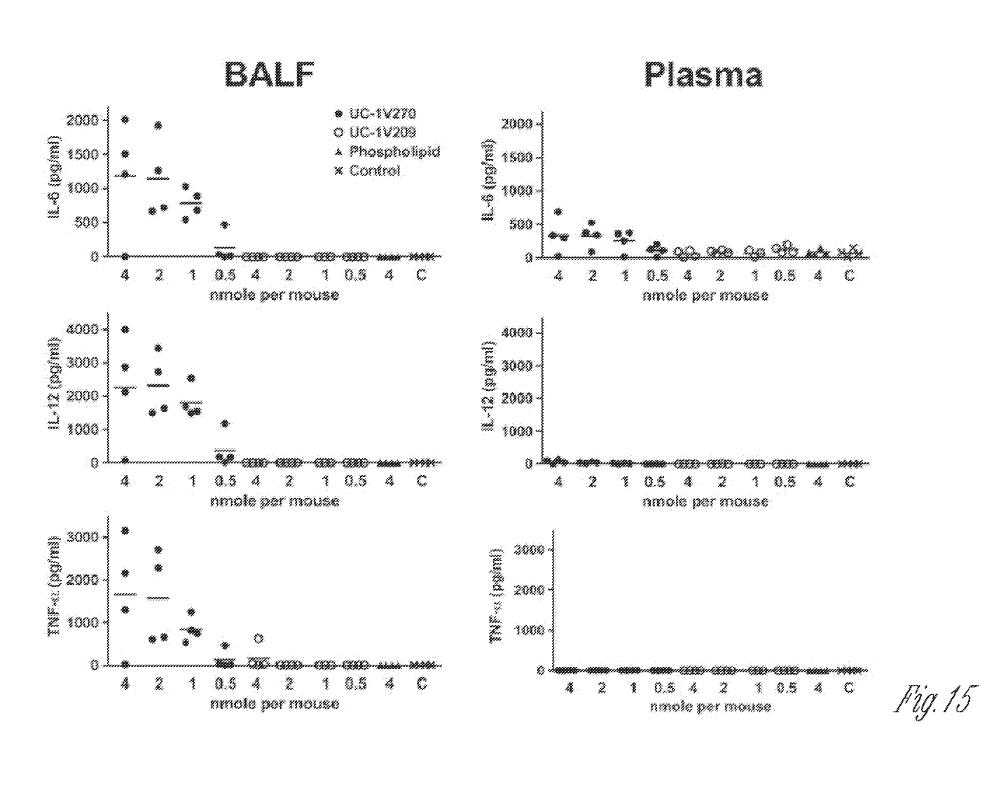

Four A/J mice were administered i.n. with UC-1V270 (nanoparticles), unconjugated TLR7 agonist (UC-1V209), phospholipid alone or a solvent control (PBS or less than 5% DMSO). BALF and plasma were collected 24 hours later and cytokine levels determined by multiplex luminex assay. UC-1V270 promoted localized cytokine release with minimal systemic side effects (FIG. 15).

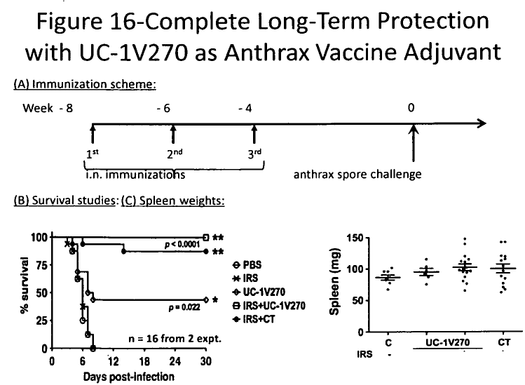

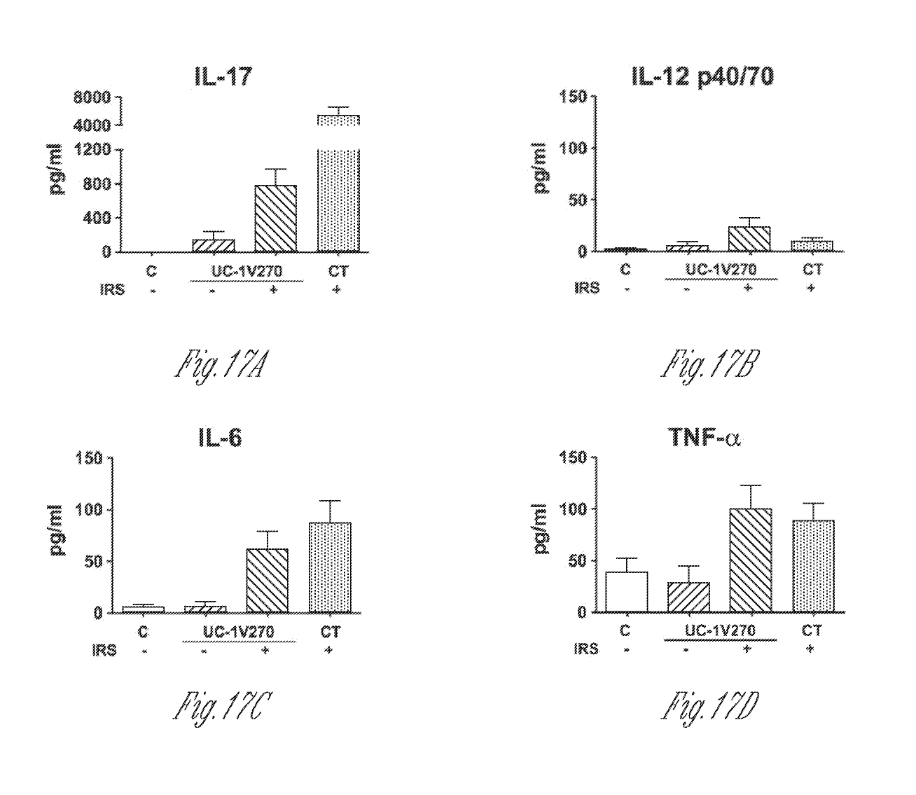

To determine the efficacy of UC-1V270 as an anthrax vaccine adjuvant, eight female A/J mice per group were administered i.n. with either PBS, IRS alone, UC-1V270 alone (nanoparticles; 1 nmol/mouse), IRS+UC-IV270 or IRS+CT (cholera toxin; 1 μg/mouse) three times at two week intends and challenged four weeks after the last immunization (FIG. 16A). Survival was followed by 30 days (FIG. 16B). Spleens from mice sacrificed at 30 days after infection were harvested and weighed (FIG. 16C).

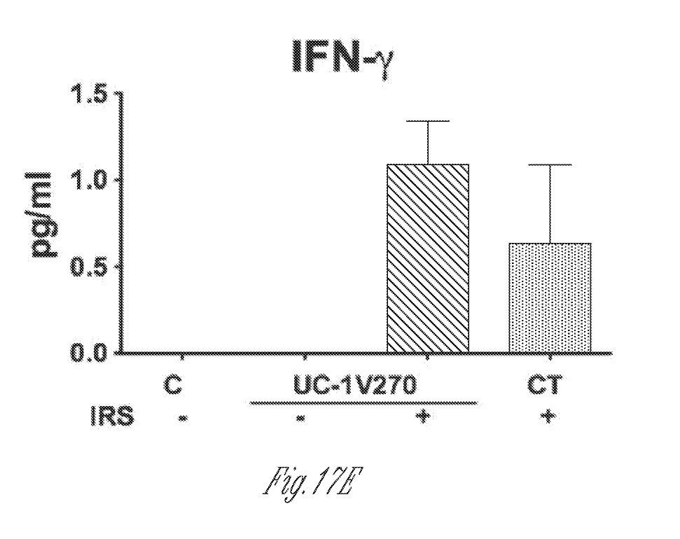

To determine the spore-specific Tₕ17 and Tₕ1 responses of surviving mice splenocytes (400,000/well) from mice that survived infection after vaccination were cultured with IRS (10⁶/well) in triplicate for 5 days, and splenocytes from uninfected non-vaccinated mice served as a control. IL-12, IL-17, TNF-α and IFN-gamma responses were measured (FIG. 17).

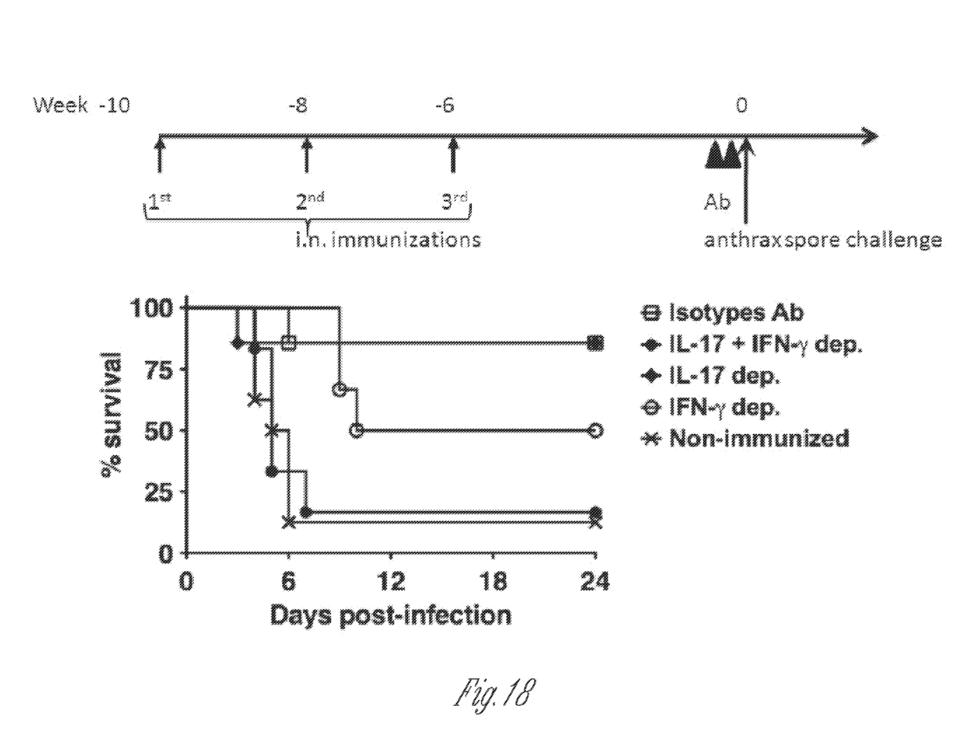

To detect whether IFN-gamma and IL-17 were important for survival, female A/J mice were administered i.n. with IRS+UC-1V270 (1 nmole/mouse) and anti-IL-17 and anti-IFN gamma antibodies were given twice daily starting one day prior to live anthrax spore challenge. The depletion of IFN-gamma and IL-17 renders immunized mice susceptible to infection (FIG. 18).

In summary, mucosal immunization with UC-1V270 and killed anthrax spores completely protected mice from pulmonary anthrax infection. Mucosal immunization induced spore-specific Th1 and Th17 cellular immune responses, and it was found that interferon-gamma and interleukin-17 were required for resistance to infection.

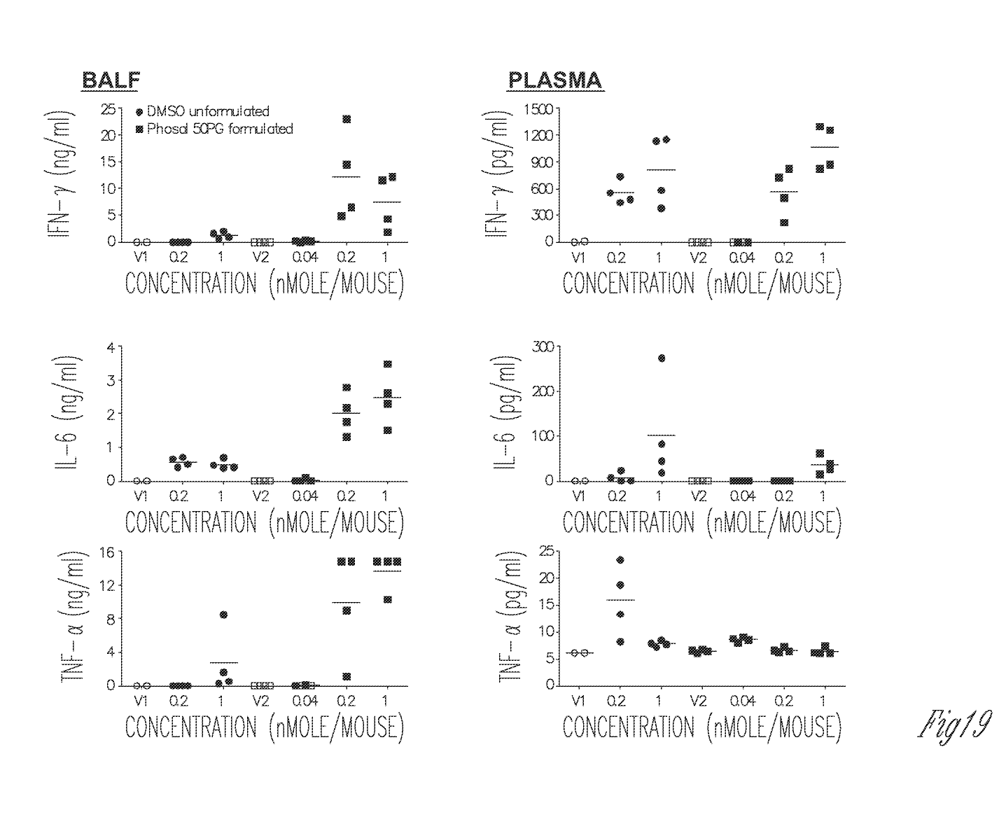

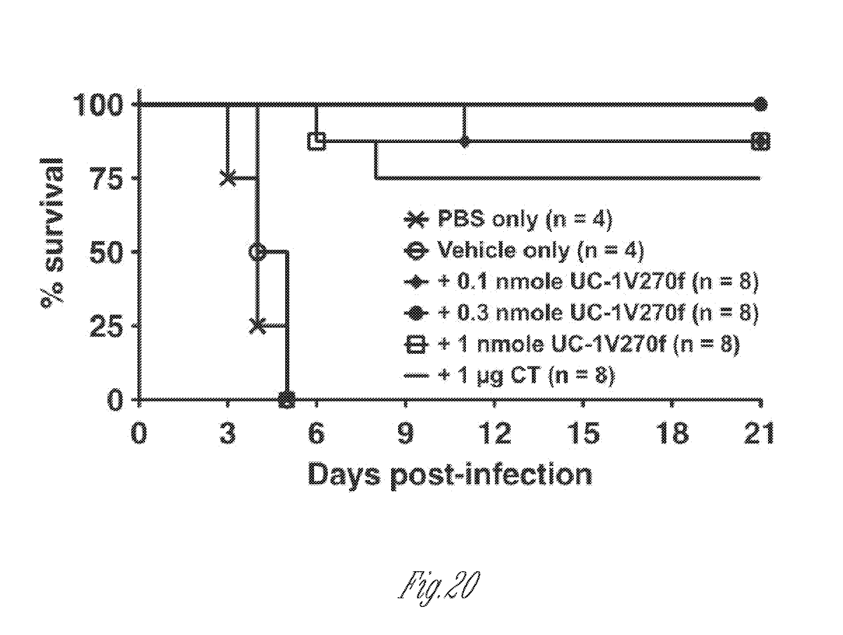

Phosal formulated 1V270 as single agent induced local cytokines with very little detectable systemic cytokine induction except IFN-g (FIG. 19). When used as an adjuvant together with irradiated spores (IRS), all 3 doses protected the animals (FIG. 20). In contrast to using 1V270 in DMSO, where a 1 nmole dose showed efficacy, the use of a much lower dose of Phosal formulated 1V270 provided significant protection.

All publications, patents, and patent documents cited in the specification are incorporated by reference herein, as though individually incorporated by reference. In the case of any inconsistencies, the present disclosure, including any definitions therein will prevail. The invention has been described with reference to various specific and preferred embodiments and techniques. However, it should be understood that many variations and modifications may be made while remaining within the spirit and scope of the invention.

1.-48. (canceled)

49. A method to augment an immune response in a mammal, comprising administering to the mammal an antigen and an effective amount of a composition comprising a compound of Formula (I): 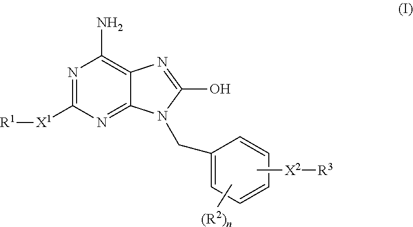
wherein X¹ is —O—, —S—, or —NR^{c}—;
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{c} is hydrogen, C₁₋₁₀alkyl, or substituted C₁₋ₘalkyl; or R^{c} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;
n is 0, 1, 2, 3 or 4;
X² is a bond or a linking group; and
R³ is a phospholipid comprising one or two carboxylic esters;
or a tautomer thereof;
or a pharmaceutically acceptable salt or solvate thereof.

50. The method of claim 49 wherein R³ comprises a group of formula 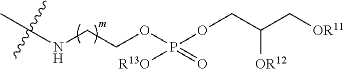
wherein R¹¹ and R¹² are each independently a hydrogen or an acyl group, R¹³ is a negative charge or a hydrogen, and m is 1 to 8, wherein a wavy line indicates a position of bonding, wherein an absolute configuration at the carbon atom bearing OR¹² is R, S, or any mixture thereof.

51. The method of claim 50 wherein m is 1.

52. The method of claim 50 wherein R¹¹ and R¹² are each oleoyl groups.

53. The method of claim 49 wherein the phospholipid of R³ comprises two carboxylic esters and each carboxylic ester includes one, two, three or four sites of unsaturation, epoxidation, hydroxylation, or a combination thereof.

54. The method of claim 53 wherein each carboxylic ester of the phospholipid is a C18 carboxylic ester with a site of unsaturation at C9-C10.

55. The method of claim 49 wherein X² is a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups.

56. The method of claim 49 wherein R³ is 1,2-dioleoyl-sn-glycero-3-phospho ethanolamine and X² is C(O).

57. The method of claim 49 wherein X¹ is oxygen.

58. The method of claim 49 wherein R¹ is hydrogen, methyl, ethyl, propyl, butyl, hydroxyC₁₋₄alkylene, or C₁₋₄alkoxyC₁₋₄alkylene.

59. The method of claim 49 wherein X¹ is O, R¹ is C₁₋₄alkoxy-ethyl, n is 0, X² is carbonyl, and R³ is 1,2-dioleoylphosphatidyl ethanolamine (DOPE).

60. The method of claim 49 wherein the antigen comprises an antigen of a microbe or a tumor-related antigen.

61. The method of claim 60 wherein the administration is effective to prevent, inhibit or treat a microbial infection.

62. The method of claim 60 wherein the microbe is a bacteria.

63. The method of claim 62 wherein the antigen comprises bacterial spores.

64. The method of claim 63 wherein the bacterial spores are from B. anthracis.

65. The method of claim 49 wherein the mammal is a human.

66. The method of claim 49 wherein the antigen and the composition are intranasally administered.

67. The method of claim 49 wherein the antigen and the composition are dermally administered.

68. The method of claim 49 wherein the antigen is administered concurrently with the composition, before the composition or after the composition.
